(19) United States
(12) Patent Application Publication
(10) Pub. No.: US 2004/0248257 A1
(43) Pub. Date: Dec. 9, 2004

(54) SPEX compositions and methods of use

(75) Inventors: Jonathan Kaye, San Diego, CA (US); Beverley Wilkinson, Wickford (GB)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA

(21) Appl. No.: 10/831,622

(22) Filed: Apr. 23, 2004

Related U.S. Application Data
(60) Provisional application No. 60/467,206, filed on Apr. 30, 2003.

(51) Int. Cl. C07H 21/04; C07K 16/18; C12N 5/06

(52) U.S. Cl. 435/69.1; 435/320.1; 435/326; 530/388.1; 536/23.5

(57) ABSTRACT

The present invention provides novel polypeptides and other factors identified as being expressed in spleen tissue and related to lymphocyte activity and the immune response. These compounds are referred to herein as SPEX compounds. Human and murine homologues of SPEX polypeptides and polynucleotides are provided including two murine alleles. The invention further provides polynucleotides encoding the polypeptides and complementary nucleic acid sequences thereof. Also provided are immunogens, expression vectors, and antibodies related to the SPEX polypeptides and polynucleotides. The present invention discloses the use of anti-SPEX antibodies in the regulation of lymphocyte activity and the regulation of the immune response.

### CROSS REFERENCE TO RELATED APPLICATION

The present application claims priority to U.S. Provisional Application Ser. No. 60/467,206 filed Apr. 30, 2003, pending.

### GOVERNMENT SUPPORT CLAUSE

This invention was made with government support under Contract AI44110 and AI31231, awarded by the National Institutes of Health. The United States Government has certain rights in the invention.

### UTILITY APPLICATION FOR U.S. PATENT

Filed Under 37 C.F.R. § 1.53(b)

### BACKGROUND OF THE INVENTION

1. Field of the Invention

The present invention is related to the fields of cell communication and signal transduction. In particular, this invention provides novel polypeptides and polynucleotides related to lymphocyte development, proliferation, and cell linage commitment; immunogens; antibodies; vectors; host cells; and other compositions; and provides methods of use thereof including for modulating lymphocyte activation and the immune response.

2. State of the Art

Certain cell surface proteins are known to play critical roles in modulating an immune response, including through cell to cell contacts and as receptors for soluble mediators. The identification and characterization of additional immune modulating proteins and methods of modulating lymphocyte development and activity remain important to the development of targeted therapies for a wide variety of conditions including autoimmunity, cancer, transplant rejection, and inflammation.

The following polynucleotides are listed in the GenBank database maintained by the National Center for Biotechnology Information (NCBI), National Library of Medicine (NLM), Rockville Pike, Bethesda, Md. 20894: expressed sequence tags AA184189 and AA177302 and bacterial artificial chromosomes (BAC) pBeloBAC11 (Incyte Genomics, Palo Alto, Calif.), PBeloBAC.26056 (Incyte), and PBeloBAC.26057 (Incyte).

### SUMMARY OF THE INVENTION

The present invention provides, in part, a novel gene encoding a cell surface protein, referred to herein as SPEX. The name “SPEX” is a contraction of the first two letters in each word of the phrase “spleen expressed” which is indicative of the high level of SPEX expression detected in spleen tissue. In certain embodiments, a substantially full length SPEX polypeptide is characterized as a signal transducing receptor expressed on lymphocytes and capable of modulating lymphocyte proliferation, differentiation, development and/or cell lineage commitment. In preferred embodiments, SPEX comprises a negative regulator of an immune response. In one example, activation of a SPEX receptor inhibits a metabolism of a lymphocyte or inhibits an immune response. In another example, antagonism of a SPEX receptor enhances a metabolism of a lymphocyte or prolongs an immune response. Certain embodiments herein provide an antibody that immunoreacts with an extracellular domain of a SPEX polypeptide and a method of use for antagonizing the SPEX receptor, thereby stimulating lymphocytes expressing the SPEX receptor.

In one embodiment, the present invention provides a SPEX polypeptide comprising a multiple domain polypeptide including an extracellular domain, a transmembrane domain, and an intracellular domain. The extracellular domain preferably includes a SPEX immunoglobulin (Ig) like domain. The transmembrane domain preferably anchors the SPEX polypeptide in a lipid bilayer, preferably a cytoplasmic membrane of a cell and, alternately a liposome. The SPEX intracellular domain preferably includes one or more, and more preferably three, tyrosine based domains.

Based, in part, on the disclosed domain structures, one embodiment provides a SPEX polypeptide comprising a type I cell surface signal transduction phosphoprotein of the immunoglobulin superfamily.

The immunoglobulin superfamily is a multigene family that includes many cell surface proteins involved in molecular recognition and adhesion. Ig like domains have a conserved core structure. Through sequence analysis, the inventors identified an Ig like domain in the extracellular portion of the SPEX receptor molecule. Examples of a preferred SPEX polypeptide include an Ig like domain having a sequence set forth in SEQ ID NO:3 of a human SPEX (hSPEX), SEQ ID NO:45 of a murine SPEX (mSPEX), or SEQ ID NO:88 of a second allele of mSPEX (referred to herein as mSPEXb). In one embodiment, a SPEX Ig like domain specifically binds a dendritic cell (e.g., through specific binding of the SPEX polypeptide to a receptor/ligand on the dendritic cell).

Phosphoproteins are another multigene family. The phosphoprotein family includes many cell surface proteins involved in molecular recognition and/or signal transduction. Phosphoproteins typically include a signaling domain that is integral to molecular recognition, phosphorylation, dephosphorylation, and signal transduction. Certain SPEX polypeptides comprise one or more tyrosine based domains. In preferred embodiments, the SPEX tyrosine based domain includes molecular recognition, phosphorylation, dephosphorylation, and/or signal transduction activities. For example, in one embodiment, a SPEX polypeptide comprising a tyrosine based domain includes a tyrosine residue capable of being phosphorylated and dephosphorylated. Examples of preferred SPEX tyrosine based domains include SEQ ID NO:7, SEQ ID NO:9, and SEQ ID NO: 11.

Based in part on the Ig like and phosphoprotein structures identified in the SPEX polypeptide and/or data disclosed herein, certain embodiments provide that SPEX receptors modulate lymphocyte function. Certain embodiments provide a method of modulating a metabolism of a lymphocyte including a SPEX receptor, comprising modulating an activity of the SPEX receptor. One embodiment provides a method of modulating a proliferation of lymphocytes that include a SPEX receptor, comprising contacting the lymphocytes with an anti-SPEX antibody that immunoreacts with an extracellular domain of the SPEX receptor. It is preferred that the proliferation of the lymphocytes, or the rate of proliferation, is increased.

In certain embodiments it is contemplated that stimulating a SPEX activity in lymphocytes that include a SPEX polypeptide will inhibit a proliferation of the lymphocytes (e.g., ligand binding of a SPEX receptor contained in a lymphocyte).

In addition to an isolated polypeptide comprising an essentially full length SPEX amino acid sequence, various domains thereof are useful in methods of producing molecules that modulate SPEX activity. For example, a SPEX extracellular domain is useful to raise antibodies, which in turn are useful to modulate SPEX activity and lymphocyte metabolism; and a SPEX intracellular domain is useful to identify a binding partner, which in turn is useful to modulate SPEX phosphoprotein activity and lymphocyte metabolism.

One embodiment of the present invention provides a SPEX polynucleotide comprising a nucleic acid sequence encoding a SPEX polypeptide, or a complement of the nucleic acid sequence. Further embodiments provide a nucleic acid for identifying a SPEX polypeptide, a nucleic acid for amplifying a SPEX polynucleotide, and a SPEX polynucleotide operably linked with a vector sequence. SPEX polynucleotides are useful, for example, in methods of producing SPEX polypeptides including in vitro and in vivo. A preferred cell for expression of a SPEX polynucleotide comprises a lymphocyte including B cells and T cells.

One embodiment provides a SPEX vector comprising a nucleic acid encoding a SPEX polypeptide, or a complement of the nucleic acid (a SPEX polynucleotide); wherein the nucleic acid is operably linked to a vector sequence. It is preferred that the vector sequence regulates a metabolism of the SPEX polynucleotide, and it is most preferred that the vector sequence regulates expression of the SPEX polynucleotide.

One embodiment provides a SPEX fusion sequence comprising a SPEX sequence operably linked to a heterologous sequence. Examples of certain useful heterologous sequences include, but are not limited to: detection tags, solubility enhancing factors, and biologically active factors.

One embodiment provides a SPEX variant sequence comprising a SPEX polypeptide or a SPEX polynucleotide, wherein the SPEX sequence includes a modification or variation as disclosed herein. It is preferred that the SPEX variant sequence includes a structural and/or a functional characteristic (i.e., an activity) of a non-modified SPEX sequence.

One embodiment provides a SPEX mutant sequence (polypeptide or an encoding polynucleotide). A preferred embodiment provides a polypeptide comprising a SPEX amino acid sequence including a mutation of a tyrosine based domain (e.g., substitution, deletion, truncation), more preferably, a mutation of a tyrosine amino acid residue of the tyrosine based domain. It is preferred that the mutation in a tyrosine based domain inhibits a SPEX signal transduction.

Another embodiment provides a host cell comprising a SPEX vector (a SPEX host cell). A SPEX host cell is useful, for example, in methods of producing a SPEX polypeptide. In another example, a SPEX host cell is useful in studies of lymphocyte metabolism and the modulation thereof including through the modulation of SPEX activation and/or inhibition.

One embodiment provides an isolated antibody, or fragment thereof, that immunoreacts with a SPEX polypeptide of the present invention. In one embodiment, the anti-SPEX antibody modulates an activity of an immune cell expressing a SPEX polypeptide. In a preferred embodiment, the anti-SPEX antibody inhibits a proliferation and/or a differentiation of a mammalian cell expressing a SPEX polypeptide, preferably a lymphocyte, and most preferably a T cell, a B-cell, and/or an antigen presenting cell (APC).

One embodiment provides a method of screening candidate molecules to identify a SPEX binding partner, comprising: contacting a candidate molecule with a SPEX polypeptide; determining whether or not the candidate molecule and the SPEX polypeptide form a binding complex, wherein the formation of a binding complex indicates that the candidate molecule is a SPEX binding partner; and continuing the screening steps until the SPEX binding partner is identified. Preferred candidate binding partner molecules include small molecule organic compounds and polypeptides. Preferred targets for the screening assay includes, but is not limited to: a SPEX polypeptide; and more preferably a SPEX extracellular domain, a SPEX intracellular domain, a SPEX Ig like domain, or a SPEX tyrosine based domain. One embodiment provides that antagonists of a SPEX polypeptide or a SPEX domain activate a lymphocyte containing SPEX receptors. Another embodiment provides that agonists of a SPEX polypeptide or a SPEX domain inhibit activation of a lymphocyte containing SPEX receptors.

One embodiment provides a method of purifying a SPEX polypeptide from a biological sample containing the SPEX polypeptide, comprising: contacting the biological sample with an affinity matrix having an anti-SPEX antibody attached to the matrix to produce an immunocomplex including the SPEX polypeptide and the antibody attached to the matrix; separating the remainder of the biological sample from the matrix; separating the SPEX polypeptide from the anti-SPEX antibody; and collecting the SPEX polypeptide.

One embodiment provides a method of modulating a metabolism, preferably a proliferation, of a lymphocyte that expresses a SPEX receptor polypeptide, comprising: contacting the lymphocyte that expresses the SPEX receptor polypeptide with an anti-SPEX antibody that immunoreacts with an extracellular domain of the SPEX receptor polypeptide, thereby modulating the metabolism of the lymphocyte. Preferred lymphocytes include B cells and T cells.

### BRIEF DESCRIPTION OF THE DRAWINGS

The drawings form a portion of the specification of the present invention.

FIG. 1 provides a diagram of substantially full-length mouse and human SPEX polypeptides (mSPEX and hSPEX, respectively) having extracellular, transmembrane (crosshatched), and intracellular domains (as labeled). The extracellular domains each include an immunoglobulin (Ig) like domain (horizontally hatched) and optionally have a cleavable signal sequence (dashed areas), wherein the arrows point to the cleavage site. The signal sequence is cleaved during post-translational processing to generate the mature SPEX polypeptide. The intracellular domains each include three tyrosine based motifs (vertically hatched). Each tyrosine based domain, in turn includes a tyrosine amino acid residue (Y, is the one-letter code for tyrosine). The embodiments shown depict that the tyrosine can be optionally phosphorylated (encircled P). The relative position of the SPEX polypeptides in a biological membrane is shown. The stippled areas indicate portions of each polypeptide between the certain functional domains.

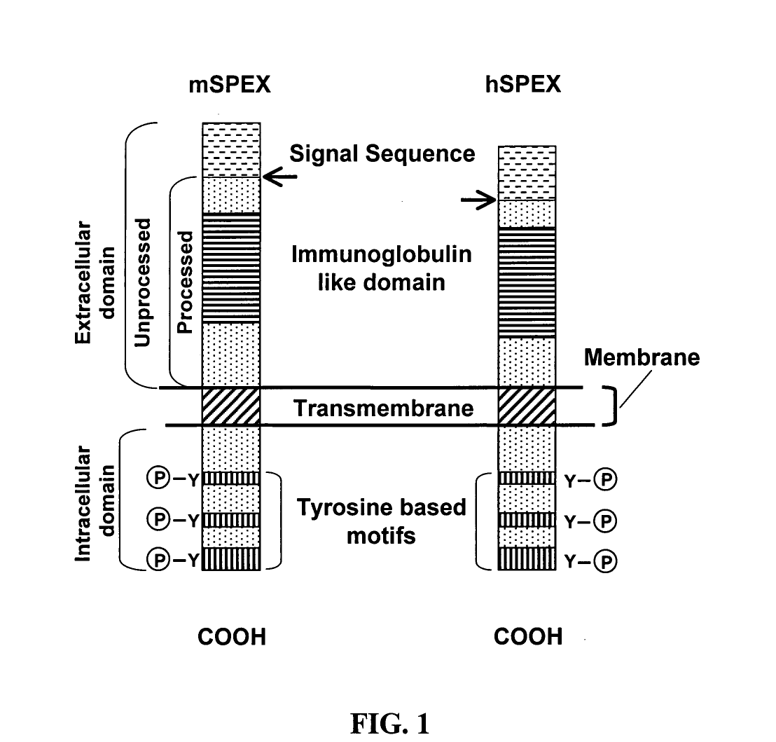

FIG. 2A provides a diagram showing embodiments of the correspondence between selected hSPEX polypeptides and the respective sequence identifier for each selected polypeptide.

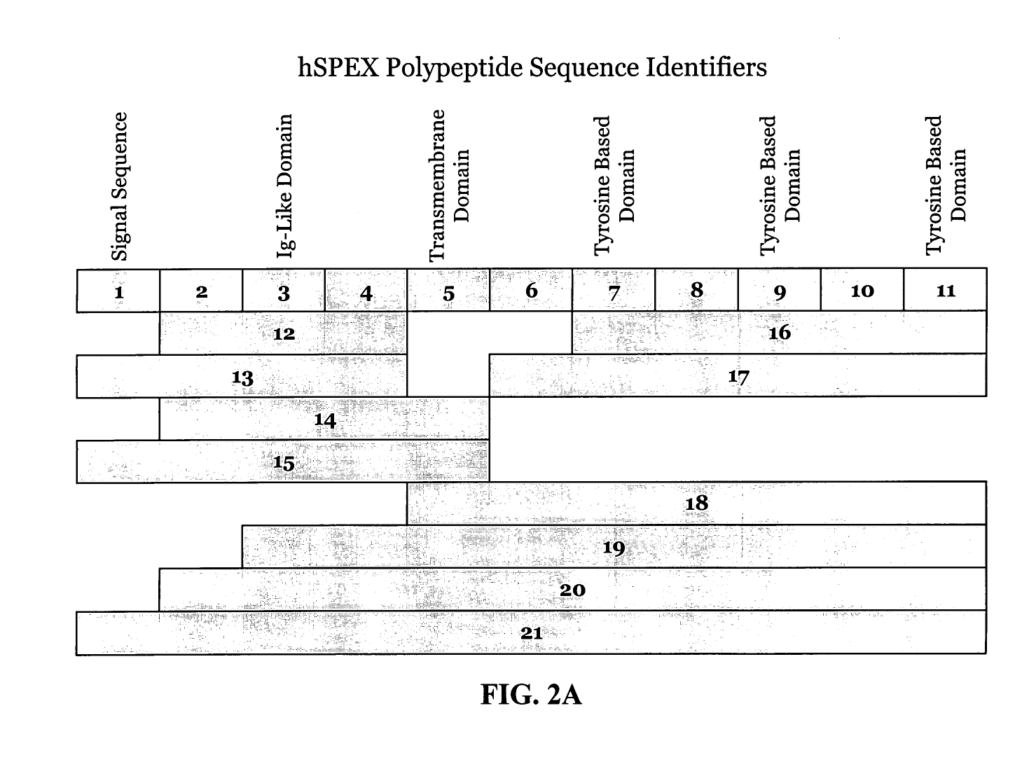

FIG. 2B provides a diagram showing embodiments of the correspondence between selected hSPEX polynucleotides and the respective sequence identifier for each selected polynucleotide.

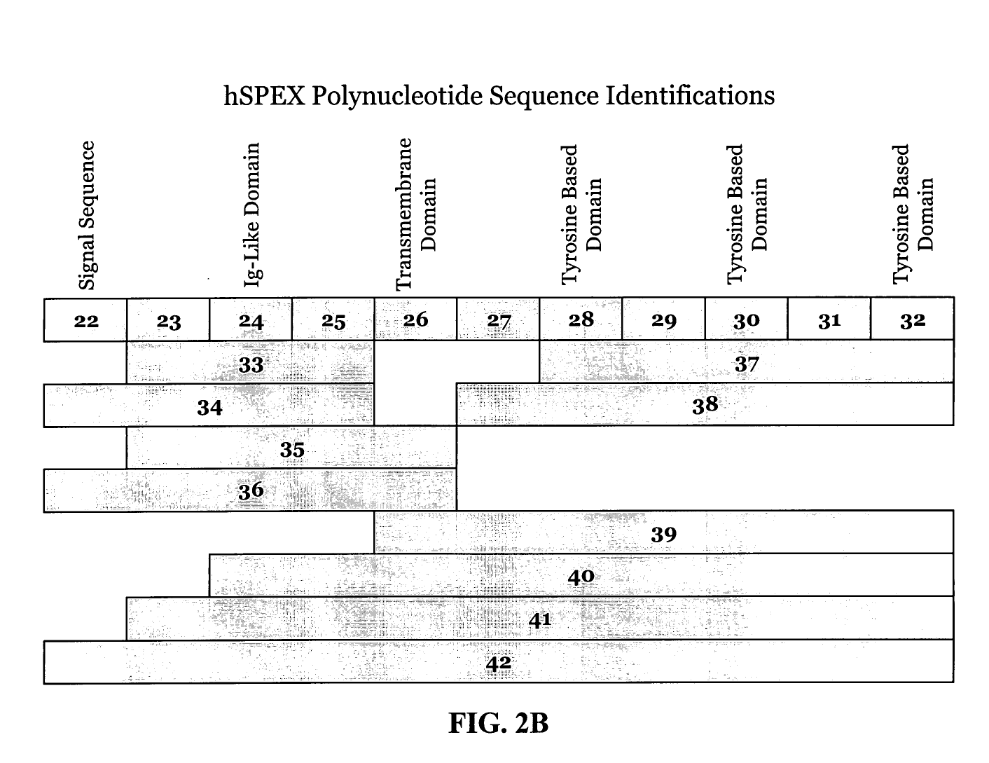

FIG. 2C provides a diagram showing certain embodiments of the correspondence between selected mSPEX polypeptides and the respective sequence identifier for each selected polypeptide.

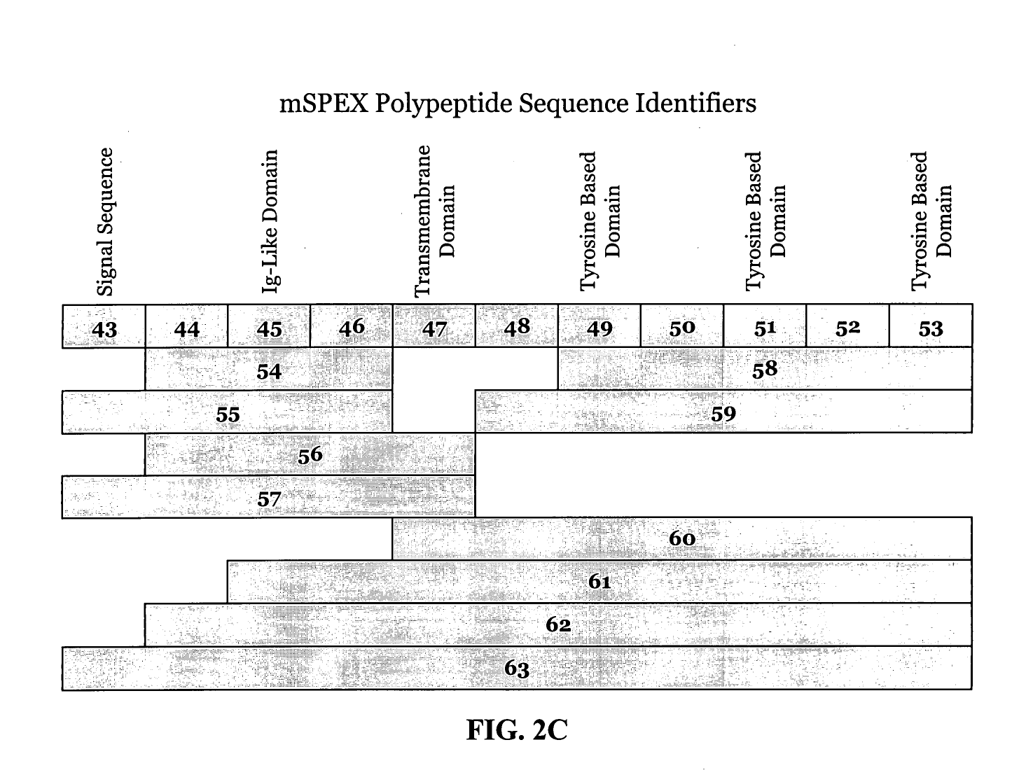

FIG. 2D provides a diagram showing certain embodiments of the correspondence between selected mSPEX polynucleotides and the respective sequence identifier for each selected polynucleotide.

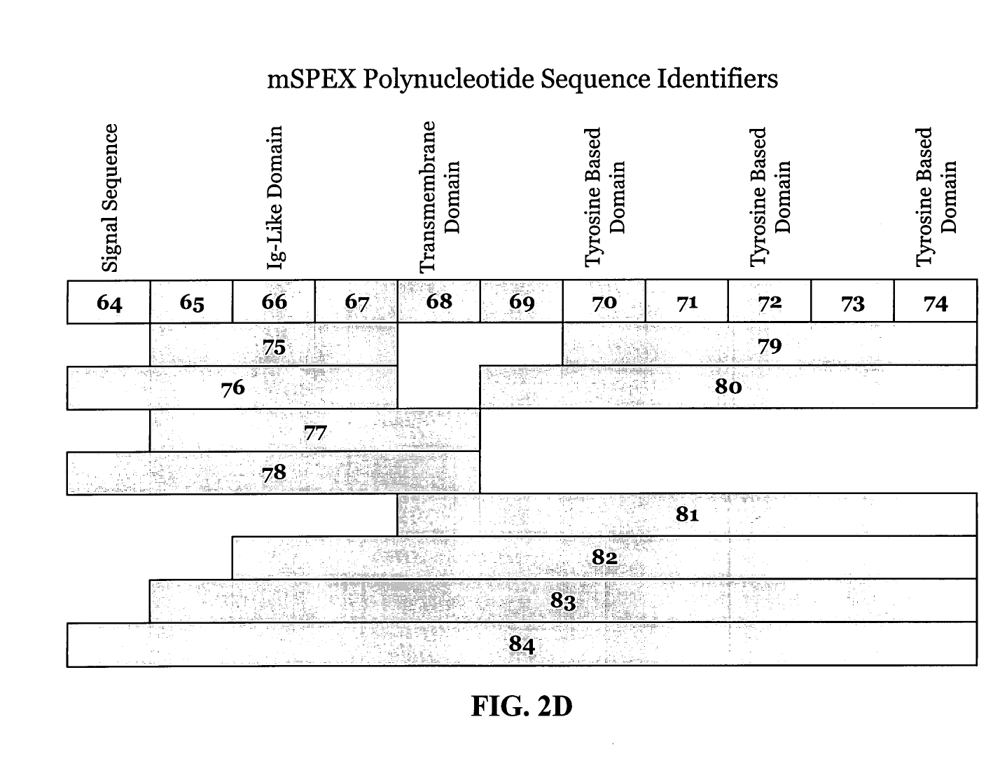

FIG. 3 provides four graphs (Panels 1-4) which demonstrate that monoclonal antibody PK18 inhibits T-cell activation. LN T cells were purified from B10.BR and BALB.K mice and cultured in 96-well plates coated with mixtures of anti-CD3 and rat Ig (open symbols) or anti-CD3 and PK18 (closed symbols). Data are expressed as the mean 3H-TdR (tritiated thymidine) incorporation of triplicate cultures (+/− standard deviation) minus counts per minute (cpm) incorporation of cultures of T cells alone (less than 1500 cpm). The incorporation of 3H-TdR into the DNA of the T cells is correlated with both proliferation of the T cells and T cell activation.

### DETAILED DESCRIPTION OF THE INVENTION

1. SPEX

The present inventors identified, in part, a messenger RNA (mRNA), the expression of which is substantially increased by stimulating thymocytes with pharmacological activators used to model the in vivo thymocyte development program (i.e., thymocytes that are stimulated to proliferate and/or differentiate, see Example 1). The present mRNA is referred to herein as “SPEX mRNA”. The inventors further identified the gene, cDNA, and polypeptide corresponding to or encoded by the SPEX mRNA. Accordingly, the present SPEX polynucleotides and SPEX expression products thereof are referred to herein, for example, as a SPEX gene, SPEX cDNA, SPEX mRNA, a SPEX antisense nucleic acid, and a SPEX polypeptide.

The present invention discloses that SPEX is expressed by both T and B cells; with expression on B cells typically being greater than expression on T cells; activation of CD4 or CD8 T cells leads to up-regulation of SPEX expression in the T cells; activation of B cells leads to down-regulation of SPEX expression in the B cells; SPEX is expressed by CD4 and CD8 T cells, T cells that express the γδTCR (T cell receptor), and CD25+CD4+ regulatory T cells; SPEX is induced in the thymus during the double positive to single positive transition; SPEX is upregulated during the pro B cell to pre B cell transition in the bone marrow and then is further upregulated during the immature and mature B cell developmental stages; SPEX is expressed on mature bone marrow-derived dendritic cells; SPEX expression is low to absent on immature bone marrow-derived dendritic cells; SPEX expression is low to absent on NK (natural killer) cells; and SPEX is expressed on antigen presenting cells in the spleen (including macrophages and dendritic cells).

In part, the present invention further provides compositions, such as: SPEX polypeptides and SPEX polynucleotides (including SPEX expression vectors), antibodies that immunoreact with SPEX polypeptides, immunogens, and SPEX host cells. The present invention also provides methods of using SPEX compositions, for example: to identify SPEX protein binding partners; to isolate SPEX from natural or recombinant sources; to modulate lymphocyte metabolism; and/or as a marker or isolation tag to distinguish, identify, and/or purify lymphocytes that express SPEX.

Definition of Purified

As used herein, the terms “isolated” and “purified” are used interchangeably and mean that the particular compound of interest is separated from other contaminating substances so as to be free (i.e., pure), or essentially free, from impurities including toxic matter such as endotoxin (optionally discounting solutes, excipients, stabilizers, buffers, salts, pharmaceutically acceptable carriers, and the like which are not necessarily contaminating substances). Accordingly, it is preferred that a polypeptide, polynucleotide, immunogen, variant, mutant, fusion (polypeptide or polynucleotide fusion), vector, host cell, antibody, and the like; of the present invention is in a purified form.

As used herein, when a purified compound of the present invention is admixed or otherwise combined with a second purified compound or substance, the resulting composition is considered a “purified” composition (i.e., having defined components).

In light of the present invention, one skilled in the art can purify a polypeptide of the present invention (including fragments thereof) using standard techniques for protein isolation and purification. A pure polypeptide generally will yield a single major band on a non-reducing polyacrylamide gel, subject to limited variation. For example, multiple bands closely spaced on an electrophoretic gel (e.g., a doublet or triplet of bands) often results from the presence of a mixed population of post-translationally modified (or other) variants of a polypeptide having a given amino acid sequence (e.g., a portion of a population of a polypeptide is phosphorylated or otherwise modified while another portion of the population does not include the modification). Alternatively, polypeptide gene products of the alleles of a gene may differ in amino acid sequence (e.g., due to a genetic mutation). The present polypeptide gene products may migrate differentially during electrophoresis, typically resulting in two bands corresponding to the two alleles found in cells with the normal complement of chromosomal loci. Additional bands are also contemplated (e.g., due to differences in phosphorylation states and/or post translational protein modification such as glycosylation). Gel bands can be examined to determine the content of the SPEX polypeptide in light of the present invention and general polypeptide sequencing techniques well known in the art such as sequencing (peptide or nucleotide) or mass spectral analysis.

Such mixed populations of the polypeptide can be further purified to homogeneity (e.g., thereby providing a pure polyeptide), if desired (e.g., based on charge, size, affinity, or other methods known in the art). Also, the population can be treated to add a modification or to remove a modification to/from the polypeptides in a population (e.g., phosphorylation can be added by a kinase or removed using a phosphatase, glycosylation can be removed by a glycosidase or added by a glycosyl transferase). The purity of the polypeptide can also be determined, for example, by amino acid sequence analysis and/or by mass spectral analysis.

In light of the present invention, one skilled in the art can purify a polynucleotide of the present invention using standard techniques for nucleic acid isolation and purification. For example, nucleic acids can be removed from proteins, lipids, and other compounds using standard nucleic acid preparation techniques. Mixed populations of nucleic acid sequences can be resolved using agarose or polyacrylamide gel electrophoresis with collection of specific bands of interest. Nucleic acids can be identified and/or purified using sequence specific probes. A sequence specific probe can be affixed to a matrix for convenient isolation of a polynucleotide of interest through affinity chromatography. Polynucleotides can also be purified using cloning techniques with selection of a single colony containing a single polynucleotide clone followed by isolation of the nucleic acid of interest from the host cell using standard techniques.

In light of the present invention, one skilled in the art can purify an antibody of the present invention using standard techniques. Certain methods include affinity purification over an antigen bound matrix (e.g., a SPEX polypeptide bound matrix) or general antibody purification over protein A matrix.

In light of the present invention, one skilled in the art can purify a host cell of the present invention using standard techniques. Certain methods include dilution plating techniques which may, or may not, include use of a selectable marker gene contained in the host cell.

In certain embodiments, a composition including a purified polypeptide, polynucleotide, antibody, host cell, or other compound of the present invention, may further include: buffer, water, salts, pharmaceutically acceptable carriers, adjuvants, fusions, labels, tags, markers, stabilizing agents, albumin, and the like, and/or protein modifications (e.g., phosphorylation or glycosylation). A purified compound of the present invention may also include one or more of such agents or be packaged with such agents and still be considered “purified”, as used herein (i.e., these agents are not necessarily contaminating substances). It is preferred that the present composition is a purified and sterile composition. For example, the sterile composition can be manufactured by combining a purified and sterile compound of the present invention with one or more sterile buffer, conditioner, carrier, bulk, binding, or stabilizing agent under sterile conditions and/or by or sterilizing the composition after combining the SPEX compound and agent.

It is preferred that a purified and/or sterile polypeptide, polynucleotide, antibody or composition of the present invention is substantially free of endotoxin or other pyrogens and undesirable irritants capable of inducing an adverse reaction when administered to humans or other mammals. The present polypeptides, polynucleotides, antibodies, or compositions can be provided in dry form (e.g., lyophilized), which form may include salts, stabilizers, etc as desired. It is preferred that the dry form embodiments are provided in a container, vessel, or vial capable of maintaining sterility and purity and, optionally, suitable for reconstitution with a reconstitution agent (see above) prior to administration to a human or other mammal. The SPEX compound may be provided in a first container in a kit packaged together with the reconstituting agent in a second container.

A SPEX Receptor

As used herein, a “substantially full length SPEX polypeptide” may be referred to as a “SPEX receptor”. It is preferred that the “SPEX receptor” includes an activity as disclosed herein, including, but not limited to: modulation of lymphocyte metabolism (e.g., activation, deactivation, or proliferation), cellular adhesion, phosphorylation, dephosphorylation (of a cytoplasmic domain). Preferred SPEX receptors include an Ig like domain, a transmembrane domain, and a tyrosine based domain (preferably an intracellular domain). Highly preferred SPEX receptors include, but are not limited to: SEQ ID NO:20, SEQ ID NO:21, SEQ ID NO:62, or SEQ ID NO:63. Alternative SPEX receptors include, but are not limited to SEQ ID NO:19 or SEQ ID NO:61. Additional alternative SPEX receptors comprise SEQ ID NO:85 or SEQ ID NO:86 operably linked to a SPEX transmembrane domain and a SPEX intracellular domain.

2. Polypeptides

Certain embodiments of the present invention provide a SPEX polypeptide of mouse, human (preferred), or other mammalian origin. An example of a human SPEX (hSPEX) polypeptide comprises an amino acid sequence set forth in SEQ ID NO:20. An example of a murine SPEX (mSPEX) polypeptide comprises an amino acid sequence set forth in SEQ ID NO:62. One embodiment provides an allele of a murine SPEX, referred to herein as mSPEXb. An exemplary sequence of the mSPEXb polypeptide comprises the amino acid sequence set forth in SEQ ID NO:86.

As used herein, the term “SPEX polypeptide” is meant to include a substantially full length SPEX polypeptide, as well as, an amino acid sequence comprising (optionally, consisting essentially of) a domain, fragment, segment, fusion, mutant, and/or a variant thereof; as described herein. It is preferred that a polypeptide of the present invention exist in a purified form.

A. Polypeptide Including A SPEX Immunoglobulin Like Domain

One embodiment of the present invention provides a polypeptide comprising an amino acid sequence including a SPEX immunoglobulin (Ig) like domain. In certain embodiments, a SPEX Ig like domain is set forth in SEQ ID NO:3, SEQ ID NO:45, or SEQ ID NO:88.

In one embodiment, a preferred SPEX Ig like domain includes a secondary and/or tertiary structure, for example, an Ig fold or a beta sandwich. In another embodiment, a SPEX Ig like domain maintains the secondary and/or tertiary structure, such as the Ig fold or the beta sandwich, when the SPEX Ig like domain is isolated or separated from a polypeptide sequence that normally contains the SPEX Ig like domain.

A SPEX Ig like domain is useful, for example, in the manufacture of an antibody that specifically binds SPEX and modulates signal transduction, lymphocyte activation, lymphocyte development, or an immune response. In another example, a SPEX Ig like domain, optionally a SPEX extracellular domain, is useful to modulate a lymphocyte function, to modulate an immune response, or to screen for SPEX binding proteins. In still another example, a SPEX Ig like domain is useful in a process of identifying agonists and antagonists of lymphocyte and/or SPEX activity.

Certain embodiments of a polypeptide comprising a SPEX Ig like domain are set forth by sequence identifier in Table 1 below. In embodiments that comprise a plurality of amino acid sequences that are not necessarily continuous, it is preferred that the sequences are operatively linked (e.g., by a peptide bond or by an amino acid sequence linker).

TABLE 1
CERTAIN EMBODIMENTS OF A POLYPEPTIDE
INCLUDING AN IG LIKE DOMAIN

| A polypeptide including: | Optionally further including | Optionally, not including |
|---|---|---|
| (domain or SEQ ID NO) | (domain or SEQ ID NO) | (domain or SEQ ID NO) |
| SPEX Ig like domain | | Transmembrane domain |
| 3 | | 5 |
| SPEX Ig like domain | | Tyrosine based domain |
| 3 | | 7, 9, or 11 |
| 3 | 7, 9, 11, 16, or 17 | 5 |
| 3 | Transmembrane domain or 5 | 7, 9, or 11 |
| 12 or 13 | Transmembrane domain or 5 | 5 |
| 12, 13, 14, or 15 | | 16 or 17 |
| 19 or 20 | | 1 |
| 21 | | |
| 45 | | 47 |
| 45 | | 7, 9, or 11 |
| 45 | 7, 9, 11, 58, or 59 | 5 |
| 45 | Transmembrane domain or 47 | 7, 9, or 11 |
| 54 or 55 | Transmembrane domain or 47 | 47 |
| 54, 55, 56, or 57 | | 58 or 59 |
| 61 or 62 | | 43 |
| 63 | | |
| 88 | | Transmembrane domain |
| 88 | | Tyrosine based domain |
| 85 or 86 | | Transmembrane domain |
| 85 or 86 | Transmembrane domain | Tyrosine based domain |
| 85, 86, or 88 | Transmembrane domain | |
| 85, 86, or 88 | Transmembrane domain and | |
| | tyrosine based domain | |
| 85, 86, or 88 | Transmembrane domain and | |
| | 7, 9, and/or 11 | |

Certain embodiments provide a polypeptide comprising (alternatively, consisting essentially of) an amino acid sequence identified in Table 1, above.

A method to determine whether a polypeptide of interest includes an Ig like domain comprises searching the Conserved Domain Database using RPS-BLAST and inputting the amino acid sequence of interest as the search query. If the polypeptide includes an Ig like domain structure, then the position and sequence of the Ig like domain is specified by the search program along with alignments of Ig like domains from other polypeptides. Preferred search parameters are: search database, all; expect 0.01; filter, low complexity; search mode, multiple hits 1-pass. The Conserved Domain Database and Search Service v1.54 tool is provided by the Computational Biology Branch, National Center for Biotechnology Information (NCBI), National Library of Medicine (NLM), Rockville Pike, Bethesda, Md. 20894. Comparison (e.g., by sequence alignment) of the candidate polypeptide to SEQ ID NO:3 or SEQ ID NO:45 is useful to determine if the candidate polypeptide is a SPEX polypeptide, fragment, or variant as disclosed herein.

B. Polypeptide Including a SPEX Tyrosine Based Domain

Another aspect of the present invention provides a polypeptide comprising a tyrosine based domain of a SPEX amino acid sequence. In one embodiment, a tyrosine based domain comprises SEQ ID NO:7, SEQ ID NO:9, or SEQ ID NO: 11. A tyrosine based domain includes a tyrosine amino acid residue. It is preferred that the tyrosine based domain comprises a phosphorylation site. It is also preferred that a SPEX tyrosine based domain is capable of being phosphorylated and desphosphorylated.

A SPEX tyrosine based domain is useful, for example, in a process of identifying a modulator of a SPEX signaling activity or lymphocyte activation. For example, it is contemplated that a SPEX binding partner that specifically binds the intracellular domain of the SPEX polypeptide, preferably at or near a tyrosine based domain motif, will interfere with and thereby inhibit phosphorylation and/or dephosphorylation of the tyrosine based domain. This is expected to modulate SPEX activity as the phosphorylation/dephosphorylation of the tyrosine based domain is contemplated to provide a pathway of SPEX signal transduction. Accordingly, one embodiment provides a method of modulating a metabolism of a lymphocyte (alternatively a SPEX signal transduction), comprising: administering a vector encoding a SPEX binding partner to the lymphocyte wherein the binding partner binds an intracellular domain of the SPEX polypeptide, preferably a tyrosine based domain. The binding partner vector expresses a polypeptide in the lymphocyte, wherein the polypeptide specifically binds the SPEX polypeptide and modulates a lymphocyte metabolism and/or a SPEX signal transduction.

Certain embodiments of a polypeptide comprising one or more SPEX tyrosine based domains are provided in Table 2, below. Table 2 also provides alternative embodiments, wherein the polypeptide includes one or more SPEX tyrosine based domains (left column), but does not include a second SPEX sequence or domain (right column).

TABLE 2
CERTAIN EMBODIMENTS OF A SPEX POLYPEPTIDE

| A polypeptide | Optionally, the polypeptide |
|---|---|
| comprising: | does not include |
| SEQ ID NO: | SEQ ID NO: |
| 7 | 1, 2, 3, 4, 5, 12, 13, 14, or 15 |
| 9 | 1, 2, 3, 4, 5, 12, 13, 14, or 15 |
| 11 | 1, 2, 3, 4, 5, 12, 13, 14, or 15 |
| 7 and 9 | 1, 2, 3, 4, 5, 12, 13, 14, or 15 |
| 7 and 11 | 1, 2, 3, 4, 5, 12, 13, 14, or 15 |
| 9 and 11 | 1, 2, 3, 4, 5, 12, 13, 14, or 15 |
| 7, 9, and 11 | 1, 2, 3, 4, 5, 12, 13, 14, or 15 |
| 16 | 1, 2, 3, 4, 5, 12, 13, 14, or 15 |
| 17 | 1, 2, 3, 4, 5, 12, 13, 14, or 15 |
| 18 | 1, 2, 3, 4, 13, or 14 |
| 49 | 43, 44, 45, 46, 47, 54, 55, 56, or 57 |
| 51 | 43, 44, 45, 46, 47, 54, 55, 56, or 57 |
| 53 | 43, 44, 45, 46, 47, 54, 55, 56, or 57 |
| 49 and 51 | 43, 44, 45, 46, 47, 54, 55, 56, or 57 |
| 49 and 53 | 43, 44, 45, 46, 47, 54, 55, 56, or 57 |
| 51 and 53 | 43, 44, 45, 46, 47, 54, 55, 56, or 57 |
| 49, 51, and 53 | 43, 44, 45, 46, 47, 54, 55, 56, or 57 |
| 58 | 43, 44, 45, 46, 47, 54, 55, 56, or 57 |
| 59 | 43, 44, 45, 46, 47, 54, 55, 56, or 57 |
| 60 | 43, 44, 45, 46, 54, or 55 |

Certain embodiments provide a polypeptide comprising (alternatively, consisting essentially of) an amino acid sequence identified in Table 2, above.

An immunoreceptor tyrosine based inhibitory motif (ITIM) is a motif known in the art to be included in certain immune cell signaling proteins, particularly immune cell membrane receptors. ITIMs participate in regulating immune responses, cell proliferation, clonal expansion, production of cytokines, cellular adhesion, and other biological activities. An ITIM is set forth by IXYXXL (SEQ ID NO:93), wherein “X” represents any amino acid.

One embodiment of the present invention provides an ITIM comprising the sequence IVYASL (SEQ ID NO:94). In one embodiment, the tyrosine based domain comprising GIVYASLNH (SEQ ID NO:9) includes the ITIM set forth in SEQ ID NO:94. Another embodiment provides a SPEX polypeptide comprising IVYASL (SEQ ID NO:94).

A signaling lymphocyte activation molecule (SLAM) associated adapter protein (SAP) is another motif known in the art to be included in certain immune cell signaling proteins. A SAP motif participates in regulating immune responses, cell proliferation, clonal expansion, production of cytokines, cellular adhesion, and other biological activities. In one example, the SAP polypeptide, SH2D1A, is known in the art to inhibit signal transduction by SLAM so that the proliferation of lymphocytes such as T cells and natural killer cells does not continue unchecked. Defects in a SAP gene at Xq25 are associated with X-linked lymphoproliferative disease (see, e.g., Buckley, R. (2000) NEJM 343:1313-1324 and Sayos J. et al. (1998) Nature 395:462-469; each article incorporated herein by reference).

One embodiment of the present invention provides a SAP binding site comprising the sequence TEYASI (SEQ ID NO:96). In one embodiment, the tyrosine based domain comprising EAPTEYASICVRS (SEQ ID NO: 11) includes the SAP set forth in SEQ ID NO:96. Another embodiment provides a SPEX polypeptide comprising TEYASI (SEQ ID NO:96).

C. SPEX Extracellular Domain

One embodiment of the present invention provides a polypeptide comprising a SPEX extracellular domain or fragment thereof. In certain embodiments, the extracellular domain comprises SEQ ID NO:3, SEQ ID NO:45, SEQ ID NO:12, SEQ ID NO:13, SEQ ID NO:54, SEQ ID NO:55, SEQ ID NO:85, SEQ ID NO:85, SEQ ID NO:86, or SEQ ID NO:88. In one embodiment, the extracellular domain further comprises a transmembrane domain (e.g., SEQ ID NO:5 or SEQ ID NO:47). Alternatively other membrane anchoring sequences known in the art may be used. Numerous transmembrane sequences are known that anchor polypeptides in plasma membrane, cell wall, cellular organelles, vesicles, liposomes, lipid rafts, and the like. One of ordinary skill in the art can use a transmembrane sequence to combine, anchor, or otherwise operably link a polypeptide of the present invention to a one of the mentioned lipid based structures, preferably a cellular plasma membrane.

D. SPEX Intracellular Domain

One embodiment of the present invention provides a polypeptide comprising a SPEX intracellular domain or fragment thereof. In certain embodiments, the polypeptide comprises SEQ ID NO:7, SEQ ID NO:9, SEQ ID NO:11, SEQ ID NO:16, SEQ ID NO:17, SEQ ID NO:58, or SEQ ID NO:59. In certain embodiments, the intracellular domain further comprises a transmembrane domain (e.g., SEQ ID NO:5 or SEQ ID NO:47). In general, other membrane anchoring sequences can be used, if desired.

E. Immunogens

Certain embodiments of the present invention provide SPEX immunogens. A “SPEX immunogen”, as used herein, is a SPEX polypeptide or a fragment thereof which is capable of eliciting an immune reaction. In one embodiment, a SPEX polypeptide or fragment thereof is useful in the manufacture of an antibody that immunoreacts with a SPEX polypeptide or fragment thereof (see above). SPEX immunogens also are useful in diagnostic and experimental assays or kits. For example, certain diagnostics and kits provide compositions and methods useful as a marker of development stage of lymphocytes, a marker of MAPK signaling pathway activation during the DP to SP stage of thymocyte development, a marker of cell type (cells that express SPEX are discussed above), or a marker or tag for purification and/or sorting of cell types based at least in part on SPEX expression.

In general, a SPEX polypeptide including six or more consecutive amino acid residues is capable of eliciting an immune response. Typically, increasing the number of amino acid residues in the SPEX polypeptide enhances the immunogenic response to the peptide. Thus, certain embodiments provide, in increase order of preference, a SPEX immunogen comprising 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 consecutive amino acids of the SPEX polypeptide. Alternatively, the SPEX immunogen comprises 6 to 9, 10 to 19, 20 to 29, 30 to 39, or 40 to 50 consecutive amino acids of the SPEX polypeptide.

In certain preferred embodiments, a SPEX immunogen comprises a SPEX polypeptide domain, such as: an external domain, a transmembrane domain, an intracellular domain, an Ig like domain, a tyrosine based domain, or a signal sequence. Each SPEX polypeptide disclosed in Table 1 and Table 2 is useful as a SPEX immunogen.

In preferred embodiments, a SPEX immunogen is purified. For example, purification of a SPEX immunogen from a longer SPEX polypeptide is preferred when the fragment is liberated from the longer SPEX polypeptide using peptidases (e.g., tyrpsin and chymotrypsin) or other polypeptide cleaving agents (e.g., piperidine). One method for manufacture of a SPEX immunogen comprises solid phase polypeptide synthesis, the general techniques of which are well known in the art. Another method comprises cellular expression and purification of a SPEX immunogen.

The purified SPEX immunogen may be combined or linked with any carrier and/or admixed with any adjuvant known in the art. The SPEX immunogenic composition thereby created is still referred to herein as a SPEX immunogen. In regard to a purified SPEX immunogen, the adjuvant or carrier is not necessarily considered a contaminant. Such carriers and adjuvants are useful to enhance the immunogenic response. Immunogenicity enhancing agents and their combination with polypeptides are well known in the art. For example, Freund's complete adjuvant, or a carrier particle, such as, keyhole limpet hemocyanin (KLH) or colloidal metals (e.g., colloidal gold).

F. Variants

Certain embodiments herein provide a polypeptide (referred to herein as a SPEX polypeptide variant) comprising an amino acid sequence having 95%, or more, sequence identity to a SPEX reference polypeptide. As used herein, a “SPEX reference polypeptide” is a polypeptide set forth by sequence identifier in Table 1 or Table 2. It is preferred that the present SPEX variant polypeptide comprises a purified SPEX variant polypeptide. In general, the greater the sequence identity of the SPEX variant polypeptide in comparison to the SPEX polypeptide, the more preferred the variant polypeptide. Accordingly, certain embodiments provide, in order of increasing preference, a polypeptide comprising an amino acid sequence having 96%, 97%, 98%, or 99% sequence identity, or more, to a SPEX polypeptide. A SPEX variant polypeptide herein includes at least one amino acid substitution, modification, addition, deletion, gap, and/or insertion when compared to a SPEX reference polypeptide. Preferred variants retain substantial biological activity compared to a SPEX reference polypeptide; accordingly, SPEX variant polypeptides are useful, in general, in most embodiments wherein a SPEX reference polypeptide is useful. The SPEX variant polypeptide is generally considered herein as an alternative embodiment to a SPEX reference polypeptide, however.

Sequence alignments and percent identity are determined in the present invention using JELLYFISH version 1.5 software by LabVelocity (LabVelocity, Inc., San Francisco, Calif.) using the following parameters: ktuple size (1), number of top diagonals (5), window size (5), gap penalty (3), scoring method (percent), weight matrix (Gonnet), gap open penalty (10), gap extension penalty (0.2), residue specific gap penalties (yes), hydrophilic gap benefit (yes), gap separation distance (8), percent of identity for delay (30.0), output order (aligned). Any or all of substitutions, deletions, insertions, additions, gaps, and inclusion of synthetic modified amino acid residues (e.g., non-naturally occurring amino acids) are meant to be included in the calculation of percent identity between two sequences. For example, substitution of an allo-isoleucine for an isoleucine or other amino acid residue in one sequence, would reduce the percent identity between sequences when aligned. The inclusion of a D-amino acid in a sequence, wherein the counterpart is an L-amino acid, will also reduce the percent identity between sequences.

Referring to a SPEX variant polypeptide including SEQ ID NO:3 (alternatively, SEQ ID NO:48 or SEQ ID NO:88), it is preferred that the polypeptide includes an Ig-like domain structure. For example, one embodiment provides a variant polypeptide comprising an amino acid sequence that is 95% or more identical to SEQ ID NO:3 (alternatively, SEQ ID NO:45 or SEQ ID NO:88) and includes an immunoglobulin like domain structure.

Procedures for making polypeptides with substitutions, deletions, insertions, additions, modified residues, gaps, etc. are routine in the art, and can be applied to the present polypeptides and variants in light of the present disclosure. One method includes expression of a polypeptide from a polynucleotide encoding such changes (e.g., using site-directed mutagenesis to modify the polynucleotide), preferably with purification of the expressed polypeptide. A preferred method of producing a variant polypeptide includes de novo synthesis of the SPEX variant polypeptide, wherein the desired alteration(s) are made during synthesis.

It is preferred in certain embodiments that amino acid substitutions are conservative substitutions. In general, substitution of a given amino acid in a wild type sequence with an amino acid having a side chain with similar characteristics has a reduced impact on the resulting structure and function of the conservatively substituted variant polypeptide. Accordingly, certain embodiments provide a conservatively modified SPEX polypeptide including ten or fewer amino acid changes (alternatively, no more than 10 amino acid changes), wherein the amino acid changes are conservative substitutions, additions, or deletions (preferably conservative amino acid substations). Fewer than 10 amino acid changes are preferred. Accordingly, in order of increasing preference, the conservatively modified SPEX polypeptide includes 9 or fewer, 8 or fewer, 7 or fewer, 6 or fewer, 5 or fewer, 4 or fewer, 3 or fewer, 2 or fewer, or 1 conservative amino acid change. Certain highly preferred conservatively modified SPEX variants include SEQ ID NO:3, SEQ ID NO:45, or SEQ ID NO:88.

It is preferred that a SPEX polypeptide comprising SEQ ID NO:7, SEQ ID NO:9, or SEQ ID NO: 11 has no more than one conservative amino acid substitution (alternatively an addition or a deletion) in SEQ ID NO:7, SEQ ID NO:9, or SEQ ID NO:11, respectively.

As used herein, the naturally occurring amino acids are grouped by their side chain characteristics for conservative substitution as follows: aliphatic side chains (G, A, V, L, I); aliphatic side chains with secondary amino group (P); aromatic side chains (F, Y, W); sulfur containing side chains (C and M, except wherein methionine is the first amino acid of a polypeptide); aliphatic hydroxyl side chains (S, T); basic side chains (K, R, H); acidic side chains (D, E, N, Q). For example, alanine may be conservatively substituted for a valine present in the non-variant (or wild type) polypeptide (the SPEX reference polypeptide). Alternatively, valine may be conservatively substituted for an alanine present in the non-variant polypeptide. In another example, proline does not have a conservative substitution. Although cysteine and methionine belong to a group of sulfur containing amino acids, it is preferred that cysteine and methionine residues are not substituted (or deleted) in SPEX Ig like domain variant polypeptides. It is also preferred that variant polypeptides including a SPEX immunoglobulin like domain have a wild type complement of cysteine residues within the Ig like domain to maintain disulfide formation and/or structural conformation of the domain.

G. SPEX Mutant Polypeptides

Certain embodiments provide a SPEX polypeptide including a mutation (including a substitution, deletion, truncation etc.), wherein the mutation inhibits a SPEX signal transduction and/or a modulation of lymphocyte metabolism. The present SPEX polypeptide is referred to herein as a “SPEX mutant polypeptide”. A SPEX mutant polypeptide differs from a SPEX polypeptide variant, for example, in that mutants having a low activity are preferred while variants having substantial activity are preferred. A “low activity” here is preferably an activity level that is 20% or less compared a reference SPEX polypeptide (preferably 10% or less and more preferably 5% or less activity). The SPEX mutant polypeptide is preferably purified.

A highly preferred SPEX mutant polypeptide comprises a mutation in a SPEX tyrosine based domain, even more preferably, the mutation comprises a substitution or deletion mutation of a tyrosine residue of the tyrosine based domain. Preferably, the SPEX polypeptide including the tyrosine based domain mutant is characterized by an inhibited SPEX signal transduction and/or a modulation of lymphocyte metabolism. A SPEX polypeptide wherein the tyrosine based domains are not mutated (i.e., wild type), preferably as set forth in SEQ ID NO:7, SEQ ID NO:9, and SEQ ID NO: 11 may be used for comparison of activity, etc.

Certain embodiments of a SPEX mutant polypeptide are set forth in Table 3 below. The numbering of the tyrosine residues of interest are provided for exemplary hSPEX and mSPEX sequences, respectively.

TABLE 3
EXAMPLARY SPEX TYROSINE BASED
DOMAIN MUTANTS

| | Residue Number | | |
|---|---|---|---|
| | Relative to hSPEX | | |
| | SEQ ID NO: 21 | | |
| | 226 | 257 | 282 |
| | Residue Number | | |
| | Relative to mSPEX | | |
| | SEQ ID NO: 63 | | |
| | 245 | 274 | 299 |
| SEQ ID NO: 21 | Y | Y | Y |
| Mutant #1 | Y | Y | F |
| Mutant #2 | Y | F | Y |
| Mutant #3 | F | Y | Y |
| Mutant #4 | Y | F | F |
| Mutant #5 | F | Y | F |
| Mutant #6 | F | F | Y |
| Mutant #7 | F | F | F |

Certain preferred SPEX mutant polypeptides comprise a dominant negative SPEX mutant polypeptide, wherein the polypeptide inhibits the activity of wild type SPEX polypeptides (e.g., by direct interaction, such as, multimer formation resulting in a defective multimer or by indirect action, such as, acting as a inhibitor for a ligand or an activator of a wild type SPEX polypeptide). Each mutant #1 through #7 in TABLE 3 above is contemplated to comprise a dominant negative SPEX mutant polypeptide. SPEX mutant polypeptides are useful, for example, in methods of modulating SPEX signaling a metabolism of a lymphocyte or an immune response. For example, the mutants may compete for a ligand of a SPEX receptor or act as a dominant negative inhibitor of SPEX activity.

H. Non-Naturally Occurring Polypeptide

In optional embodiments, a polypeptide of the present invention comprises a non-naturally occurring polypeptide. Examples of non-naturally occurring polypeptides include: (a) a polypeptide that includes an amino acid not found in nature or in the organism of interest, such as a synthetic amino acid or amino acid mimetic; (b) a polypeptide that includes a chemical moiety attached to the polypeptide that is not associated with the polypeptide in nature, such as a radioactive isotope or a fluorescent marker; (c) a polypeptide that comprises a purified amino acid sequence wherein the full sequence occurs in nature, e.g., a purified truncated sequence; (d) a polypeptide that does not include a portion of a naturally occurring polypeptide, such as an internal deletion; (e) a fusion polypeptide including two or more amino acid sequences (the same or different sequences) joined together, wherein the amino acid sequences are not found joined together in nature; (f) or a purified polypeptide removed from the milieu of naturally-occurring substances normally associated with the polypeptide in nature. The SPEX polypeptide may be purified, for example, from a mammalian cell or from other cell types (e.g., bacterial, yeast, insect, plant) using techniques known in the art, including recombinant cloning in a host cell and purification therefrom. The polypeptide may be chemically synthesized, the techniques of which are well known in the art.

I. Polypeptide Fusions

One embodiment provides a polypeptide comprising at least a first SPEX amino acid sequence operably linked to a second polypeptide, wherein the first and the second polypeptides are not found linked in nature (referred to herein as a “SPEX polypeptide fusion). SPEX amino acid sequences useful for making SPEX fusions are disclosed herein (e.g., the polypeptides disclosed in Table 1 and Table 2, the SPEX variants disclosed herein, and the SPEX mutants disclosed herein). The terms “SPEX fusion” and “SPEX fusion sequence” refer generically to SPEX fusion polypeptides and to SPEX fusion polynucleotides. A SPEX fusion polypeptide of the present invention is isolated and preferably is purified. In general, the polypeptide components that comprise a SPEX fusion can be linked in any desired order. SPEX fusion polypeptides are useful, for example, to promote (enhance or increase) solubility, purification, expression, detection, selection, and antigenicity; of a SPEX polypeptide of interest.

One embodiment provides a polypeptide comprising a first SPEX polypeptide operably linked to a heterologous polypeptide, wherein the heterologous polypeptide is different from the first SPEX polypeptide, and wherein the heterologous polypeptide and the first SPEX polypeptide are not normally found operably linked in nature. For example, “heterologous polypeptide” includes SPEX and non-SPEX sequences and sequences from the same or a different species.

In certain embodiments, the operable linkage is cleavable. For example, a SPEX fusion polypeptide may include a peptidase cleavage site for separating the SPEX polypeptide and the heterologous polypeptide. Cleavable peptide sequences are well known in the art and can be incorporated into a SPEX fusion polypeptide operable linkage (e.g., by cloning or de novo synthesis of the desired amino acid sequence), in light of the present disclosure. Enzymes for such cleavage include tyrpsin, enterokinase, tissue plasminogen activator (tPA), factor Xa, furin, and others.

A preferred operable linkage of a SPEX fusion polypeptide comprises a polypeptide bond (amide bond) joining each polypeptide of the fusion to one or more other polypeptides. Preferably, each polypeptide of a fusion is linked to another polypeptide of the fusion by a single polypeptide bond.

A preferred method for making a SPEX fusion polypeptide is to make a contiguous polypeptide using genetic engineering techniques (i.e., expression of a polynucleotide engineered to combine the coding regions for the respective polypeptides by a peptide bond or amino acid linking sequence). A SPEX fusion polypeptide can also be manufactured by de novo chemical synthesis (e.g., solid phase polypeptide synthesis). Optionally, the polypeptides of the fusion can be operably linked by an amino acid spacer sequence. Spacer sequences are useful, for instance, to separate functional domains in a fusion polypeptide (e.g., to avoid steric hindrance). Such spacer sequences are known in the art and typically including multiple glycine, alanine, and/or proline residues.

Alternatively, chemical cross linkers are also useful for operably linking polypeptides. Chemical crosslinking of polypeptides is known in the art and can be applied to making a SPEX fusion polypeptide in light of the present invention. Common reactive or functional groups in chemical crosslinking reagents include: iuccinimidyl esters, maleimides, and iodoacetamides (kits for chemical crosslinking are commercially available, for example, from Molecular Probes, Eugene, Oreg.). Common reactive groups of polypeptides include amino, carboxyl, sulfhydryl, aryl, hydroxyl and carbohydrates. Certain specific chemical crosslinking reagents include: DSP (Dithiobis(succinimidylpropionate)), DTSSP 3,3′-Dithiobis(sulfosuccinimidylpropionate), DSS (Disuccinimidyl suberate), BS³ (Bis(sulfosuccinimidyl) suberate), DST (Disuccinimidyl tartrate), Sulfo-DST (Disulfosuccinimidyl tartrate), EDC (1-Ethyl-3-(3-Dimethylaminopropyl)carbodiimide Hydrochloride), DTME: Dithio-bis-maleimidoethane, SPDP (N-succinimidyl-3-(2-pyridyldithio) propionate).

Other useful chemical crosslinkers include heterodifunctional crosslinkers which contain two or more different reactive groups and typically allow for sequential conjugations with specific groups of proteins, minimizing undesirable polymerization or self conjugation. Heterodifunctional crosslinkers which react with primary or secondary amines include imidoesters and N-hydroxysuccinimide (NHS)-esters such as succimidyl 4-(N-maleimidomethyl) cyclohexane-1-carboxylate (SMCC) and succimidyl-4-(p-maleimidophenyl)-butyrate (SMPB). Crosslinking reagents which react with sulfhydryl groups include maleimides, haloacetyls and pyridyl disulfides. Carbodiimide crosslinking reagents couple carboxyls to primary amines or hydrazides, resulting in formation of amide or hydrazone bonds. One widely used carbodiiumide crosslinking reagent is 1-ethyl-3-(3-dimethylaminopropyl)-carbodiimide hydrochloride (EDAC), which is commonly used to couple carboxylic acids to amines and is an example of a zero-length crosslinking reagent. These crosslinking reagents are available from Pierce Chemical Co. (Rockford, Ill.) and Sigma (St. Louis, Mo.), for example. Photoreactive crosslinking reagents are also typically heterobifunctional crosslinking reagents. Upon UV illumination, these reagents react with nucleophiles or form C—H insertion products. Various methods for introducing reversible, substantially irreversible, and cleavable chemical crosslinks between polypeptides are also well known in the art and can be used to make SPEX fusion polypeptides in light of the present disclosure.

A noncovalent interaction between two molecules that has very slow dissociation kinetics can also function as an operable linkage. For example, avidin, streptavidin, NEUTRAVIDIN biotin-binding protein and CAPTAVIDIN biotin-binding protein can operably link up to four molecules of a biotinylated target through high affinity, but non-covalent binding.

In addition the heterologous sequence optionally comprises any desirable biologically active factor, for example; an antibody, a cytokine (e.g., interleukins, interferons, NF-κB, an IL-2R chain (Tac antigen)), a peptide hormone (e.g., EGF, TGFα, TGFβ), a chemokine, a kinase, a phosphatase, a membrane translocation sequence or factor, an nuclear translocation sequence, a membrane anchor, a toxin (e.g., ricin, the active portion of diphtheria toxin), a marker or identifying tag, and the like.

One embodiment provides a polypeptide comprising a SPEX polypeptide operably linked to a heterologous polypeptide, wherein the heterologous polypeptide is substantially soluble in aqueous solution, preferably more soluble in aqueous solution than the SPEX polypeptide. Operably linking a SPEX polypeptide with a more soluble heterologous polypeptide is desirable, for example, to assist in expression, binding studies, and in raising antibodies. A preferred substantially soluble heterologous polypeptide comprises a constant region of human IgG1, more preferably the human IgG1 hinge CH2, and CH3 domains. In one embodiment, a solubility promoting heterologous polypeptide comprises SEQ ID NO:97. Another embodiment provides a polypeptide comprising a SPEX polypeptide set forth in SEQ ID NO:3, SEQ ID NO:45, SEQ ID NO:88, SEQ ID NO: 12, SEQ ID NO:54, or SEQ ID NO:86 operatively linked to a heterologous polypeptide including a constant region of human IgG1; preferably the human IgG1 hinge, CH2, and CH3 domains. Protein A binds the constant region of IgG1 with high affinity. Accordingly, a SPEX-human IgG1 fusion can be purified using protein A affinity chromatography.

Examples of other contemplated solubility enhancing polypeptides include thioredoxin (TRX), glutathione S-transferase (GST), Protein A, DsbA, and Escherichia coli maltose-binding protein (MBP) or soluble fragments thereof. Another example includes the HAT epitope which is a 19-amino-acid sequence from the chicken lactate dehydrogenase protein (BD Biosciences Clontech, Palo Alto, Calif.). The HAT sequence of non-adjacent histidine residues possesses less overall charge than tags with consecutive His residues, such as the 6×His tag. As a result, HAT polypeptide fusions exhibit increased solubility compared to the 6×His tag fusions while still possessing strong affinity for immobilized metal ions. The binding characteristics of the HAT sequence allow both imidazole gradient and pH gradient purification of proteins under native conditions (e.g., at approximately neutral pH (pH 7), as well as under denaturing conditions. An alternative embodiment provides a SPEX polypeptide operably linked with a non-peptide organic solubility factor, preferably linked by a covalent bond to the SPEX polypeptide.

Examples of contemplated detectable labels or markers include: antibody recognized moieties, for example, FLAG epitope, DYKDDDDK (SEQ ID NO:101); c-myc epitope, EQKLISEEDL (SEQ ID NO:102), visualization markers (e.g., green fluorescent protein, luciferase, biotin/avidin), enzymatic markers (e.g., horseradish peroxidase), radiolabels (e.g., ¹²⁵I, ¹⁴C, ³H, ³³P, ³⁵S, and ³²P), and combinations thereof (e.g., radiolabeled and enzyme labeled antibodies).

Examples of contemplated purification tags or aids include: GST, MBP, TRX, calmodulin binding peptide (CBP), 6-His tag (or poly-His), FLAG, c-myc tag, radiolabels, fluorescent markers, and hemagglutinin (HA). These, and other moieties provide convenient purification of the corresponding SPEX fusion on immobilized glutathione, maltose, phenylarsine oxide, calmodulin, and metal chelate resins, respectively. Such moieties are commonly utilized in commercially available purification systems. A fusion protein may also be engineered to contain a proteolytic cleavage site located between the SPEX polypeptide sequence and the heterologous polypeptide sequence, so that a SPEX polypeptide may be cleaved away from the heterologous moiety following purification. A variety of commercially available kits are available to facilitate making a fusion polynucleotide including a desired insert, expressing a product from the insert, and purifying the product. These kits may provide vectors that place a cleavable linker between the product of interest and the heterologous polypeptide of the fusion such that the portions of the fusion product can be cleaved and purified (e.g., the CREATOR compatible expression systems, Clontech). In light of the present disclosure, these kits can be used to make a SPEX fusion sequence (polynucleotide and polypeptide).

J. Polypeptide Manufacture

In light of the present disclosure, a SPEX polypeptide of this invention can be made using a variety of techniques well known in the art for preparing or making a polypeptide. For example, by purification from natural sources (e.g., lymphocytes or spleen or thymus tissue). In another example, a SPEX polypeptide can be made through production in a host cell including in bacterial (e.g., K12), eukaryotic, yeast, insect, plant, mammalian, Chinese hamster (e.g., CHO), murine, and human cells (e.g., using transfer of a recombinant SPEX expression system). In still another example, SPEX polypeptide is made using synthetic de novo methods. In preferred embodiments, the SPEX polypeptide is purified using methods known in the art for protein separation and purification, which methods are possible for isolating or purifying a SPEX polypeptide in light of the present invention. For example, in light of the present invention, one of ordinary skill in the art is able to use an anti-SPEX antibody disclosed herein to isolate or purify a SPEX polypeptide including specific fragments and domains thereof through affinity separation.

Examples of useful amino acids (and nucleotides) are provided in the World Intellectual Property Organization (WIPO) Handbook on Industrial Property Information and Documentation, Standard ST.25: Standard for the Presentation of Nucleotide and Amino Acid Sequence Listings in Patent Applications (1998), including Tables 1 through 6 in Appendix 2; hereinafter referred to as “WIPO Standard ST.25 of 1998”, incorporated herein by reference.

3. Polynucleotides

One embodiment of the present invention provides a polynucleotide (referred to herein as a “SPEX polynucleotide”), comprising a nucleic acid sequence encoding a SPEX polypeptide, or a complement of the nucleic acid sequence. The complement of the nucleic acid sequence is capable of hybridizing to the encoding nucleic acid sequence. It is preferred that the SPEX polynucleotide comprises a purified polynucleotide.

SPEX polynucleotides are useful, for example, in manufacturing SPEX polypeptides (e.g., in vitro or cellular polypeptide expression wherein the SPEX polypeptide is preferably purified). SPEX polynucleotides are also useful in methods that include administering a SPEX expression product to a cell, wherein the SPEX polynucleotide includes an element for expression. A complement of the nucleic acid sequence encoding a SPEX polypeptide (a “SPEX complement”) is useful, for example, in assays or diagnostic kits suitable for detecting a SPEX expression product. The present diagnostic kit, in turn, is useful to detect lymphocyte type or in methods of cell purification or sorting.

In one embodiment, the SPEX polynucleotide comprises a mammalian SPEX polynucleotide, preferably a murine SPEX (mSPEX) polynucleotide, and, more preferably, a human SPEX (hSPEX) polynucleotide. One embodiment provides a polynucleotide comprising a nucleic acid sequence encoding a polypeptide set forth in Table 1 or Table 2 (or, alternatively, a SPEX variant polypeptide as disclosed above).

It is preferred that a polynucleotide of the present invention comprises a purified polynucleotide, which purified form is substantially free of contaminating substances (discounting solutes, excipients, stabilizers, buffers, and the like) and removed from the milieu of substances with which SPEX occurs in nature. It is preferred that a purified SPEX polynucleotide is substantially free of single stranded oligonucleotide sequences used for polymerase chain reaction (PCR) amplification and/or sequencing.

Table 4 provides examples of correspondence between SPEX domains and certain preferred polynucleotides along with polypeptides encoded by the polynucleotides. The respective sequences are set forth by sequence identifier.

TABLE 4
CERTAIN EMBODIMENTS OF SPEX POLYNUCLEOTIDES

| | hSPEX | | mSPEX | |
|---|---|---|---|---|
| | Nucleic Acid | Amino Acid | Nucleic Acid | Amino Acid |
| SPEX Sequence/Domain | SEQ ID NO | SEQ ID NO | SEQ ID NO | SEQ ID NO |
| Signal Sequence | 22 | 1 | 64/91 | 43/87 |
| Ig Like Domain | 24 | 3 | 66/92 | 45/88 |
| Transmembrane | 26 | 5 | 68 | 47 |
| Tyrosine based Domain | 28 | 7 | 70 | 7 |
| Tyrosine based Domain | 30 | 9 | 72 | 9 |
| Tyrosine based Domain | 32 | 11 | 74 | 11 |
| Extracellular Domain (Processed) | 33 | 12 | 75/90 | 54/86 |
| Extracellular Domain (Unprocessed) | 34 | 13 | 76/89 | 55/85 |
| Extracellular (Processed) and | 35 | 14 | 77 | 56 |
| Transmembrane Domains | | | | |
| Extracellular (Unprocessed) and | 36 | 15 | 78 | 57 |
| Transmembrane Domains | | | | |
| Tyrosine Based Region | 37 | 16 | 79 | 58 |
| Intracellular Domain | 38 | 17 | 80 | 59 |
| Transmembrane & Intracellular | 39 | 18 | 81 | 60 |
| Domains | | | | |
| Ig-Like Domain through COOH | 40 | 19 | 82 | 61 |
| SPEX (no Signal Sequence) | 41 | 20 | 83 | 62 |
| SPEX (including Signal Sequence) | 42 | 21 | 84 | 63 |

The embodiments in TABLE 4, above having more than one sequence identifier (e.g., “45/88” listed for a murine SPEX Ig like domain) refers to distinct specific alleles identified in the particular organism.

As defined herein, the maximum length of a SPEX polynucleotide that includes the adjacent genomic 5′ and 3′ regions with which a SPEX gene is normally associated in nature is 40,000 basepairs (discounting vector sequences, see below). To fall within the bounds of the present invention, a genomic clone of SPEX polynucleotide sequences must necessarily be no more than 40,000 basepairs (bases for single stranded nucleic acids). It is preferred that the genomic clone, having 40,000 basepairs or fewer, separated from other adjacent genomic sequences normally associated with a SPEX gene found in nature. As further defined herein, the minimum length of a SPEX polynucleotide is 18 residues (e.g., a polynucleotide consisting essentially of a nucleic acid encoding a polypeptide set forth in SEQ ID NO:94 or SEQ ID NO:96).

In preferred embodiments, a SPEX polynucleotide features a preferred length range. For example, one of ordinary skill in the art is aware that for each application or system using a polynucleotide, there are typical length or length ranges that make efficient use of the polynucleotide in the application. Accordingly one of ordinary skill in the art is able to select a nucleic acid length best suited to an application of choice, in light of the present invention. Table 5 below provides examples of embodiments using a SPEX polynucleotide and preferred lengths of SPEX polynucleotides for selected applications or systems.

TABLE 5
EXEMPLARY LENGTH RANGES OF SPEX
POLYNUCLEOTIDES

| | Preferred range for |
|---|---|
| | SPEX polynucleotide: |
| | (nucleotides or base |
| Application or system of use: | pairs as appropriate) |
| SPEX polynucleotide | 18 to 40,000 (minimum to maximum) |
| (discounting vector sequences) | more preferably 99 to 15,000 |
| | more preferably 150 to 7,000 |
| | most preferred 600 to 2,000 |
| SPEX insert for plasmid vector | preferably 24 to 15,000 |
| | more preferably 150 to 5,000 |
| | most preferred 630 to 1,500 |
| SPEX insert for viral vector | preferably 24 to 25,000 |
| | more preferably 180 to 10,000 |
| | most preferred 810 to 1,800 |
| SPEX polynucleotide | preferably 500 to 40,000 |
| insert in yeast artificial | |
| chromosome (YAC), | more preferably 10,000 to 35,000 |
| bacterial artificial | |
| chromosome (BAC), | still more preferably 20,000 to 30,000 |
| or cosmid | |

Referring to Table 5, above, length ranges are in nucleotides when referring to single stranded polynucleotides and basepairs when referring to double stranded nucleotides. The ranges of preferred SPEX polynucleotide lengths disclosed in Table 5 are suggested for enhanced efficiency relative to each composition or application, but are not necessarily absolute. In general, transformation efficiency of a given vector system decreases with increasing size of the insert used in the vector.

One embodiment provides a polynucleotide, referred to herein as a “SPEX fusion polynucleotide”, comprising a nucleic acid sequence that encodes a polypeptide including at least a first SPEX amino acid sequence operably linked to a second polypeptide, wherein the first and the second polypeptides are not found linked in nature. For example, a polynucleotide comprising a nucleic acid sequence that encodes a SPEX fusion polypeptide. The present embodiment is especially useful for producing a SPEX fusion polypeptide in quantity.

Certain preferred SPEX fusion polynucleotides include a nucleic acid sequence encoding one or more of a first amino acid sequence set forth by: SEQ ID NO:3, SEQ ID NO:45, SEQ ID NO:88, SEQ ID NO:12, SEQ ID NO:13, SEQ ID NO:54, SEQ ID NO:55, SEQ ID NO:85, or SEQ ID NO:86, operatively linked to the a polypeptide set forth in SEQ ID NO:97. The present fusion polynucleotides are useful in the production of an external domain of the SPEX polypeptide, wherein the fusion is substantially soluble in aqueous solution, preferably more soluble than the SPEX polypeptide alone.

Compositions and methods are well known in the art for cleaving, hybridizing, and ligating segments of polynucleotide sequences, which compositions and methods are useful for the production of a SPEX fusion polynucleotide in light of the present invention. If desired, a SPEX fusion polynucleotide may include a restriction site for separating the segments of the SPEX fusion polynucleotide.

A. Intervening Sequence

In one embodiment a polynucleotide further comprises an intervening sequence (e.g., an intron) operably linked with a SPEX nucleic acid sequence. An intron operably linked with the SPEX polynucleotide may be desirable, for example, to enhance transcription and to enhance stability of a mRNA expression product, particularly in a mammalian expression system. Typically the intron is operably linked between a first and a second segment of the SPEX nucleic acid sequence. Accordingly, one embodiment of the present invention provides a polynucleotide comprising a SPEX polynucleotide and an intron sequence, wherein the intron is operably linked to the nucleic acid upstream of the nucleic acid sequence. A preferred intron sequence is selected from one or more SPEX genomic intron sequences. Optionally, the intron sequence is selected from one or more heterologous intron sequence or a combination of a SPEX intron and a heterologous intron.

Examples of useful intron sequences include a SV40 small-t antigen intron and an intron A from the CMV IE gene. These intron sequences are available commercially from Promega (Madison, Wis.) and Invivogen (San Diego, Calif.), respectively. It is preferred that the junctions between the coding sequence and the intron sequence (i.e., the splice junctions) comprise suitable splice sites for removal of the intronic sequence during processing (e.g., during a splicing reaction such as the processing of hnRNA to mRNA). In certain embodiments the SPEX polynucleotide includes any intron having suitable splice junctions.

B. Degenerate Genetic Code Variant

One of ordinary skill in the art is aware of the degeneracy of the genetic code and is therefor able to use degenerate codons to manufacture multiple nucleic acid sequences encoding the same SPEX polypeptide in light of the present invention. For a table of the standard genetic code and use thereof, as well as, the symbols for common amino acids and the abbreviations or codes thereof, see Biochemistry, 3rd Edition, Stryer Ed., Freeman Publisher (1988) pages 91-115 and inside back cover, incorporated herein by reference. A suitable SPEX polynucleotide can be formulated, in light of the present invention, based upon the desired SPEX polypeptide sequence to be expressed and knowledge of the standard genetic code.

C. Sequence Modification Techniques

Modifications to a SPEX sequence may be made, for example, during chemical synthesis of the polymer (including polynucleotide and polypeptide synthesis) or through mutagenesis (variegation) of a polynucleotide and, optionally, expression of an encoded polypeptide variant. A preferred method of altering a polynucleotide sequence is through site directed mutagenesis. Methods for chemical synthesis, mutagenesis, site directed mutagenesis, and expression of a polynucleotide are well known in the art. Methods for confirming production of the desired polynucleotide and polypeptide sequence are also known in the art. These methods can be applied to making and confirming SPEX sequence variants, in light of the present disclosure and knowledge in the art.

D. Vectors

In certain embodiments, it is desirable that a SPEX polynucleotide further comprises a vector sequence. Accordingly, the present invention provides a purified polynucleotide, comprising a vector sequence operatively linked to a nucleic acid sequence encoding a SPEX polypeptide, or a complementary sequence thereto. The nucleic acid encoding the SPEX polypeptide, or the complement thereof, are referred to herein as a “SPEX insert”. As used herein, a “SPEX vector” includes a “vector sequence” operably linked with a “SPEX insert”. SPEX vector sequences are useful, for example, to express a SPEX mRNA, a SPEX polypeptide, or a SPEX hybridizing sequence (e.g., an “antisense” nucleic acid). SPEX expression products may be labeled with a detectable label if desired. For example, the detectable label can be incorporated during synthesis. SPEX vectors are also useful as convenient cloning tools (e.g., shuttle vectors) or for production of SPEX nucleic acids in commercial amounts. Suitable SPEX inserts include SPEX polynucleotides listed in the tables and sections herein, complements thereof, or encode SPEX polypeptides disclosed herein (e.g., the SPEX polypeptides disclosed in Table 1 and Table 2, above).

A preferred use for a SPEX vector is in administering a SPEX expression product to a cell. For example, a SPEX polypeptide can be administered to a cell by introducing the SPEX vector into the cell, wherein the cell synthesizes the SPEX expression product using cellular machinery. Expression of a SPEX polypeptide is useful, for example, to modulate a metabolism of a lymphocyte, an immune response, and/or a SPEX signal transduction.

As used herein, the SPEX insert may include untranslated regions, cloning sequences, intervening sequences, splicing sequences, or other sequences (limited by the disclosures above) in addition to the encoding region. It is preferred that a SPEX vector is purified. A SPEX vector includes circular and linear nucleic acids.

The SPEX vector sequence preferably includes one or more control elements for modulating the expression, replication, or other activity of the vector and/or the SPEX insert. Control elements include one or more of a(n): affinity tag, branch point, cellular localization signal, enhancer, inducer, internal ribosome entry site (IRES), intron, Kozak sequence, polyadenylation site (poly A site), promoter, purification tag, repressor, selectable marker, signal sequence, silencer, splice acceptor, splice donor, start codon (initiator codon, translation start site, ATG), stop codon, TATA box, terminator, and transcription start site (e.g., Shine-Dalgarno sequence). The control elements may be contained in the vector sequence and/or the SPEX insert (optionally one or more control elements can be located on another separate vector and act in cis). Numerous vector systems are available from commercial, academic, and other sources and can be readily operably linked with a SPEX insert using techniques well known in the art, in light of the present disclosure.

In certain preferred embodiments the SPEX vector is capable of being expressed. Expression of a SPEX product from a SPEX polynucleotide, preferably of a SPEX vector, includes: transcription of an RNA from a DNA, translation of a polypeptide from an RNA or both transcription and translation. In one embodiment, expression comprises transcription of the SPEX nucleic acid sequence encoding the SPEX polypeptide forming a SPEX mRNA. It is preferred that the nucleic acid sequence encoding the SPEX polypeptide is “in frame” as contained in the vector. Most expression vectors are available, or easily adapted, for incorporation of an insert in the proper reading frame for synthesis of the desired polypeptide. A table of degenerate codons provides the preferred correspondence between amino acid residues and the nucleotide codon(s) that specify each amino acid residue during polypeptide synthesis and is useful to predict or confirm with sequencing analysis that a SPEX insert is in a desirable reading frame.

I. Promoters and Enhancers

It is preferred that a SPEX vector includes a promoter and/or enhancer for increasing the expression of the SPEX insert. In general, promoters and enhancers are control elements that modulate expression of a transcript from a template nucleotide. The basic distinction between enhancers and promoters is operational and not absolute. Typically, promoter elements are located around the initiation site for RNA polymerase II (or other polymerase) and orient the direction of transcription. Promoter elements usually include 7 to 20 bases of nucleic acid and may contain one or more recognition sites for transcriptional activators and/or repressors. Typically a module in each promoter functions to position the start site for RNA synthesis. A well known example of this is the TATA box. In general, enhancer elements are capable of modulating (typically stimulating) transcription at a distance from the transcriptional start site. Enhancers may be located on the same nucleic acid as the start site of transcription (cis) or on another nucleic acid (trans). In embodiments including a promoter, it is understood that the term “operably linked” means that the promoter is in a location and/or orientation in relation to the insert to control RNA polymerase initiation and expression of the transcript.

Promoter and/or enhancer elements may regulate the frequency of transcriptional initiation. Typically, these are located in the region 30-110 bp upstream of the transcriptional start site, although a number of promoters are known to contain functional elements downstream of the start site as well (e.g., within the transcribed nucleic acid). The spacing between promoter elements is usually flexible, so that promoter function is preserved when elements are inverted or moved relative to one another. For example, in the tk promoter (from the herpes simplex virus (HSV) thymidine kinase gene), the spacing between promoter elements can be increased up to 50 bp apart before activity begins to decline. Another example includes the lac operon. Depending on the promoter, it appears that individual elements can function either cooperatively or independently to activate transcription.

In certain embodiments, the particular promoter(s) that is(are) employed to control the expression of a SPEX insert is not believed to be critical, so long as it is capable of expressing the polynucleotide in the targeted environment at desirable or sufficient levels for the intended embodiment (e.g., at a detectable level or over-expressed compared to natively produced SPEX). Thus, for example, for expression in a human cell, it is preferable to position the polynucleotide coding region adjacent to and under the control of a promoter that is capable of being expressed in a human cell. Generally speaking, such a promoter might include either a human or viral promoter. Useful viral promoters include those from HSV tk, SV40 early transcription units, cytomegalovirus (CMV), mouse mammary tumor virus-long terminal repeat (MMTV-LTR), murine sarcoma virus (MSV-LTR), and Rous sarcoma virus (RSV-LTR). Useful promoters derived from human or other mammalian genes include those from β-actin and elongation factor 1-α (EF-1α). Numerous vectors are available that have promoter and enhancer elements suitable for expression or modulated expression in a variety of environments. Certain preferred environments include human cells, preferably lymphocytes, and more preferably T-cells or B-cells.

By employing a promoter with well-known properties, the level and pattern of expression of a SPEX polynucleotide can be optimized. For example, selection of a promoter which is substantially more active in specific cells, such as tyrosinase (melanoma), alpha-fetoprotein and albumin (liver tumor), CC10 (lung tumor) and prostate-specific antigen (prostate tumor) will permit tissue specific expression of a SPEX polynucleotide. Accordingly, in preferred embodiments, the promoter and/or vector is selected to drive transcription that is optimal in a particular desired environment.

II. Non-Cellular Expression Environment

Non-cellular expression environments, commonly known as “in vitro expression (systems)”, typically comprise cellular lysates or factors for transcription and/or translation of a template polynucleotide contained in a reaction vessel. Preferably, the in vitro expression system is at least partially refined to remove inhibitors and/or increase reagent concentrations, and more preferably, substantially purified. In vitro expression systems are commercially available (e.g., SP6 transcription kit, T7 transcription kit, SINGLE TUBE PROTEIN SYSTEM 3, and RED NOVA LYSATE KIT each available from Novagen, Madison, Wis. and the RAPID TRANSLATION SYSTEM (RTS) available from Roche, Indianapolis, Ind.). A non-cellular expression system is preferred in the event that a SPEX expression product of interest is toxic to the host cell in a cellular expression environment. One embodiment provides a SPEX expression vector adapted for use in an in vitro expression system.

III. SPEX Host Cells

Useful cellular expression environments include prokaryotic and eukaryotic host cells. The term “host cell” refers to a cell including a SPEX polynucleotide, preferably a SPEX vector, wherein the SPEX polynucleotide is introduced to the cell through the hand of man to form a SPEX host cell (e.g., a recombinant, non-naturally occurring, SPEX polynucleotide transferred into a cell for the purpose of expression). Accordingly, one embodiment provides a host cell including a SPEX vector. The present “host cell” is referred to herein as a “SPEX host cell”. Preferably, the SPEX vector comprises a SPEX expression vector and preferably is capable of producing a SPEX expression product.

In preferred embodiments, the SPEX host cell does not have SPEX expression in the absence of a transformed SPEX polynucleotide suitable for expression (e.g., a recombinant SPEX expression vector). Cells that express SPEX without the introduction of a SPEX polynucleotide by the hand of man have a “native SPEX expression” as used herein. SPEX expression from a SPEX polynucleotide transformed into the cell by the hand of man refers herein to “an exogenous SPEX expression” or a “recombinant SPEX expression”. Exogenous SPEX expression is meant to include SPEX expression from a SPEX polynucleotide incorporated into the genome of the host cell in a process directed by the hand of man. In optional embodiments, the SPEX host cell may have a native SPEX expression; however, it is preferred that the expression product of the exogenous SPEX polynucleotide is in greater abundance than the native SPEX product (i.e., it is preferred that the recombinant SPEX polynucleotide is over expressed).

Certain embodiments provide a polynucleotide including a vector sequence operably linked with a SPEX insert, wherein the vector sequence includes one or more control elements for expression of the SPEX insert in a host cell; preferably one or more of a bacteria cell, a yeast cell, an insect cell, or a mammalian cell. Numerous vectors and host cells useful for expression of the SPEX insert in light of the present invention are available from academic, commercial, and other sources. Certain additional examples of useful host cells include: Escherichia coli (E. coli) and the K12 strain of E. coli (bacterial cells); the yeast cells Schizosaccharomyces cerevisiae (S. cerevisiae), S. pombe, and Pichia pastoris (P. pastoris); the insect cells Spodoptera frugiperda 9 (Sf9), Sf21, Trichoplusia ni (commonly referred to as High Five cells), and S2 (drosophilae); and the mammalian cells include HEK-293 (human kidney), Chinese hamster ovary (CHO), Bowes Melanoma (human melanoma), HeLa (ovarian carcinoma), CHU2 (human oral tumor), and HBEC-90 (human brain endothelium), 33.1.1 (mouse pre-B cell), K46 (mouse B lymphoma), TR2 (mouse oligodendritic). Certain preferred mammalian cells include: lymphocytes, T-cells, B-cells, immature T-cells, immature B-cells, differentiated T-cells, differentiated B-cells, and natural killer cells. These cells, and others, are available from e.g., Invitrogen, Novagen, CLONTECH, and the American Type Culture Collection (ATCC, Manassas, Va.).

In certain embodiments, it is desirable to express the SPEX insert in more than one host cell. Accordingly, in certain embodiments it is preferred to use a vector system having control elements suitable for expression of the SPEX insert in multiple host cell types. For example, the TRIEX MULTISYSTEM EXPRESSION VECTORS (CALBIOCHEM, La Jolla, Calif.) includes optimized transcription and translation signals for expression in E. coli, baculovirus, and mammalian cell types.

Expression in Bacteria

Useful bacterial cell cloning vectors for cloning with a SPEX insert include: pUC8, pUC9, pBR322, pBR329, pBC-SK, pBC-KS, LAMBDA ZAP II (available from academic sources, Promega (Madison, Wis.), or Stratagene (La Jolla, Calif.). It is preferred that host cells include an origin of replication capable of functioning in the given cellular environment. For example, bacterial vectors may include a E. coli origin of replication (ori).

The pET vector series (Novagen (Madison, Wis.), Promega, Stratagene) contains the T7 promoter and the T7 gene 10 translation initiation signals useful for driving high level expression of a SPEX insert in bacterial cells that include the T7 factors. The pET vector system is made inducible by transfection into bacterial cells that lack T7 RNA polymerase, for example, BL21 (DE3). Expression is induced by transfection with a vector for expressing the missing T7 polymerase or by removing repression of native T7 expression.

The pL Expression System (Invitrogen, Carlsbad, Calif.) also provides tight inducible transcriptional control of bacterial expression systems. The pL vectors include the strong pL promoter and are capable of driving expression of a SPEX insert. The pL promoter is controlled by the lambda cI repressor protein which is expressed in the E. coli host. The cI repressor gene is engineered into the bacterial chromosome under the control of the tightly-regulated trp promoter. Expression of the SPEX polynucleotide can be induced by the addition of tryptophan.

The pBAD vectors (Invitrogen) are useful for inducible expression of an operably linked SPEX insert driven from the araBAD promoter which can be modulated to stimulate or repress transcription of the SPEX insert by the addition of arabinose or glucose, respectively, to the culture medium. Additional vectors that include one or more control elements for driving expression in bacterial systems include: Trc/Tac promoter vectors (CLONTECH, Palo Alto, Calif.), Lambda PR promoter vectors (Pharmacia, Peapack, N.J.), and Phage T5 promoter based vectors (QIAGEN, Valencia, Calif.). Embodiments provide a SPEX insert operably linked with each vector or any vector, in general, including one or more control elements for expression a product of a SPEX insert in a bacterial cell.

Expression in Yeast

Certain embodiments provide a polynucleotide including a vector sequence operably linked with a SPEX insert, wherein the vector sequence includes one or more control elements for expression of the SPEX insert in a yeast cell. It is preferred that a SPEX expression vector capable of expression in yeast includes a yeast origin of replication (e.g., ColE1).

The pESC vectors (Stratagene) include GAL1 and GAL10 control elements for expression in yeast cells, preferably S. cerevisiae. The pESC vector set includes expression one or more different expression products using selectable features including: a FLAG epitope; a c-myc epitope (tag); and HIS3, TRP1, LEU2, or URA3 selectable markers.

The pYES vector set (Invitrogen) includes a GAL1 promoter and is useful for expressing a SPEX insert in yeast cells, preferably S. cerevisiae. The ESP Yeast Protein Expression and Purification System (Invitrogen) includes a nmtl promoter of S. pombe and is useful for expressing a SPEX insert in yeast cells, preferably S. pombe. The ESP vector set includes expression one or more different expression products using selectable features including: a FLAG epitope; a c-myc epitope (tag); and HIS3, TRP1, LEU2, or URA3 selectable markers. Certain ESP vectors further include a glutathione s transferase (GST) peptide tag for convenient purification of yeast expressed SPEX-GST fusion polypeptides by GST affinity chromatography. The SpECTRA S. pombe Expression System (Invitrogen) includes additional vectors useful for construction of a SPEX vector capable of expression in yeast. The Pichia pastoris Expression System (Invitrogen) includes vectors useful for construction of a SPEX expression vector capable of expression in yeast, preferably P. pastoris, and bacteria, preferably E. coli.

Expression in Insect Cells

Certain embodiments provide a polynucleotide including a vector sequence operably linked with a SPEX insert, wherein the vector sequence includes one or more control elements for expression of the SPEX insert in an insect cell.

The DES (Drosophila Expression System, Invitrogen) is optimized for expression of a SPEX insert expression in Drosophila cells, preferably S2 cells. The DES vectors pAc5.1/V5-His and pMT/V5-His provide for expression promoted by an Ac5.1 or V5 promoter, respectively and include a His tag for detection and/or purification. The pMT/BiP/V5-His vector further provides the BiP Drosophila secretion signal sequence for secretion of an expressed polypeptide.

The INSECTSELECT System (Invitrogen) is useful for expression of a SPEX insert in a broad range of insect cell lines including Sf9, Sf21, HIGH FIVE, and S2; preferably for the expression of secreted proteins.

Expression in Mammalian Cells

Certain embodiments provide a polynucleotide including a vector sequence operably linked with a SPEX insert, wherein the vector sequence includes one or more control elements for expression of the SPEX insert in a mammalian cell, preferably a human cell.

Adenoviral and retroviral vectors are useful for expressing a SPEX insert in mammalian cells. The ViraPower Adenoviral Expression System, for example, includes E1 and E3 deleted, pAd-DEST adenovirus based vectors having a human CMV expression promoter or without promoter for convenient insertion of a promoter of choice. Packaging of the adenovirus particles is described in the manufactures literature (Invitrogen). The ViraPower Lentiviral Expression System, in another example, is optimized for stable transduction of dividing and non-dividing cells (Invitrogen).

The pShooter vectors (Invitrogen) include EF-1α or CMV promoters and may further include a signal sequence capable expressing a SPEX polypeptide and of directing the expressed SPEX polypeptide to a specific cellular compartment. For example, the pEF/myc/cyto vector includes an EF-1α promoter and a c-myc tag. A signal sequence is not included with the present vector for obtaining cytoplasmic expression. The pEF/myc/nuc vector includes an EF-1α promoter, a c-myc tag, and a nuclear localization sequence (from SV40) capable of directing an expressed SPEX polypeptide to the nucleus of a mammalian cell. The pEF/myc/mito vector includes an EF-1α promoter, a c-myc tag, and a mitochondrial localization sequence (from COX VIII cDNA) capable of directing an expressed SPEX polypeptide to the nucleus of a mammalian cell. Another set of pShooter vectors include a CMV promoter which replaces the EF-1α promoter. The pShooter vectors are capable of driving expression in a broad range of mammalian cell types include most human cells and tissues.

The pSG5 vector (Stratagene) is capable of driving expression of a SPEX insert in a broad range of cell types including mammalian (including human) and bacterial cell types. Mammalian expression is driven by an SV40 early promoter and bacterial expression is driven by a T7 promoter. The pSG5 vector preferably further includes an ori, ampicillin resistance gene, and a phage f1 origin (e.g., allows rescue of ssDNA for use in mutagenesis and sequencing).

The pIRES-hrGFP-1 vector (Stratagene) is capable of expressing a SPEX insert and a marker gene (in this example, a humanized green fluorescent protein (hrGFP)) using a CMV promoter. The vector includes an internal ribosome entry site (IRES) providing for expression of both the SPEX insert and the marker gene from a single expressed mRNA species. Thus, the SPEX and the marker gene are expressed as a fusion mRNA and expressed as separate polypeptides. Other SPEX expression vectors may further include an IRES, if desired.

In certain embodiments, the SPEX vector construct further includes a segment encoding another expression product. For instance, the vector may include a selectable marker, detectable label, and/or purification label. Examples include a segment encoding: amp, neo, hygro, zeo, puro, mycophenolic, kan, enhanced green fluorescent protein (EGFP), enhanced cyan fluorescent protein (ECFP), enhanced yellow fluorescent protein (EYFP), DsRed2, HcRed1, myc tag, FLAG tag, and GST tag. In certain embodiments, the vector is selected to provide the label positioned to form an operably linked fusion with the SPEX insert. Certain vectors further provide an internal ribosome entry site (IRES). See, e.g., U.S. Pat. No. 4,937,190 to Palmenberg et al., incorporated herein by reference. In one embodiment, an IRES facilitates the expression of two proteins (at least one comprising the SPEX polypeptide) in animal cells using a single-transcript vector (STV).

Cellular expression of SPEX typically involves introduction of the SPEX expression construct into a cell of choice wherein cellular factors drive expression. Method for introducing expression vectors into a cell are known in the art (e.g., calcium phosphate precipitation, lipid based transfection, peptide based membrane translocation, electroporation, and the like) See U.S. Pat. No. 6,312,956 to Lane and U.S. Pat. No. 6,165,720 to Felgner et al., each patent incorporated herein by reference in its entirety.

Table 7 below provides preferred embodiments of SPEX vectors and Table 6 provides preferred features and uses for these SPEX vector constructs.

TABLE 6
PREFERRED SPEX EXPRESSION VECTORS

| | | | Vector Backbone Description | | |
|---|---|---|---|---|---|
| SPEX Vector | Size | | Backbone | | RE sites |
| Designation | (kb) | SPEX Insert | Designation | Backbone Source | Insert/Backbone |
| pSPEX-YFP | 5.7 | 957 bp SPEX gene | pEYEP-N1 | CLONTECH | Bgl II/Bam H1 |
| | | (SEQ ID NO: 106) | | | |
| SPEXYFP- | 6.8 | 1.7 bp fragment of | pBABEpuro | Nucleic Acids | Not1 fill Bgl II/ |
| BABEPURO | | pSPEX-YFP | | Res. (1990) | SnaBI, BamHI |
| | | (SEQ ID NO: 107) | | 18: 3587-3596 | |
| pSPEX-Bgl II | 5.4 | 1276 bp SPEX gene | pCR 2.1 | Invitrogen | Bgl II/Bam H1 |
| | | (SEQ ID NO: 109) | TOPO | | |
| pSPEX-Bgl II- | 6.4 | 1276 bp SPEX gene | pBABEpuro | Invitrogen | Bgl II/Bam H1 |
| BABEpuro | | (SEQ ID NO: 109) | | | |
| pSPEXCAL | 6.7 | 957 bp SPEX gene | pCAL-C | Stratagene | Bgl II/Bam H1 |
| | | (SEQ ID NO: 106) | | | |
| pSPEXCAL2 | 6.4 | 486 bp SPEX | pCAL-C | Stratagene | Bam H1/Bam |
| | | extracellular | | | H1 |
| | | domain (SEQ ID | | | |
| | | NO: 110) | | | |
| pSPEXCAL3 | 6.2 | 464 bp SPEX | pCAL-C | Stratagene | Not I, Bgl II/ |
| | | fragment | | | Hind III |
| | | (SEQ ID NO: 111) | | | |
| pSPEXdeltaN | 4.5 | 609 bp SPEX gene | pCR 2.1 | Invitrogen | BamHI, EcoRI/ |
| | | (internal domain) | TOPO | | BamHI, Eco RI |
| | | (SEQ ID NO: 108) | | | |
| pSPEXIgTOPO | 4.45 | 538 bp SPEX | pCR 2.1 | Invitrogen | Bgl II/Bam H1 |
| | | external domain | TOPO | | |
| | | (SEQ ID NO: 112) | | | |
| p1017SPEX | 9.3 | 1276 bp SPEX gene | p1017 | Int. Immunol. | Bgl II/Bam H1 |
| | | (SEQ ID NO: 109) | | (1990) 2: 173-180 | |
| SPEXYFP- | 6.5 | 1722 bp SPEX SEQ | pMIGR1- | Blood (1998) | Not I, Bgl II/ |
| MIGR1 NOT | | (SEQ ID NO: 107) | NOT Linker | 92: 3780 | Hind III |
| pSPEX-Ig | 8 | 538 bp SPEX | pL-Selectin- | | Kpn I/Bam HI |
| | | external domain | Fc | | |
| | | (SEQ ID NO: 112) | | | |
| pSPEXIg.B | 4.5 | 537 bp mSPEXb | pCR 2.1 | Invitrogen | Bgl II/Bam H1 |
| TOPO | | external domain | TOPO | | |
| | | (SEQ ID NO: 113) | | | |

Each of the vectors in Table 6 above are produced using standard cloning methods known to one of ordinary skill in the art. “Backbone Designation” refers to the “vector sequence”.

TABLE 7
PREFERRED USE OF CERTAIN SPEX VECTORS

| SPEX Vector Designation | Preferred Use |
|---|---|
| pSPEX-YFP | Expression of SPEX-YFP fusion. |
| | YFP is the yellow fluorescent |
| | protein detection marker. |
| SPEXYFP-BABEPURO | Retroviral vector for SPEX expression. |
| pSPEX-Bgl II | Cloning of SPEX polynucleotides. |
| pSPEX-Bgl II-BABEpuro | Retroviral vector for SPEX expression. |
| pSPEXCAL | Bacterial expression of SPEX polypeptides. |
| pSPEXCAL2 | Bacterial expression of SPEX polypeptides. |
| pSPEXCAL3 | Bacterial expression of SPEX polypeptides. |
| pSPEXdeltaN | Vector encoding the intracellular |
| | domain of SPEX. |
| pSPEXIgTOPO | Cloning and Expression of a SPEX |
| | External Domain-IgG1 chimera. |
| p1017SPEX | Expression of SPEX polypeptides |
| | in thymocytes with Lck, a |
| | thymocyte specific promoter. |
| SPEXYFP-MIGR1 NOT | Retroviral vector for SPEX expression. |
| | Expression of SPEX in lymphocytes. |
| pSPEX-Ig | Expression of a SPEX external |
| | domain-IgG1 fusion. |
| | Provides a SPEX external domain |
| | with enhanced solubility. |
| pSPEXIg.B TOPO | Cloning of SPEX external domain to |
| | make an in frame fusion with IgG1. |

4. Antibodies

One embodiment of the present invention provides an antibody that immunoreacts with a SPEX polypeptide. The term “antibody” is meant to include any form of antibody, including intact antibodies molecules and/or an immunologically active portion of an antibody molecule. Antibodies and active fragments thereof are well known in the art, for example: IgG, IgM, IgE, polyclonal, monoclonal, Fab, Fab′, F(ab′)₂, F(v), single chain antibody (SCA), single chain Fab, humanized, hybrid, and the like). Antibodies provided in the present invention immunoreact with one or more portions of a SPEX polypeptide. It is preferred that the antibody is isolated. It is still more preferred that the antibody comprises a recombinant, chimeric, or otherwise non-naturally antibody occurring.

Anti-SPEX antibodies are useful, for example, in modulating SPEX signal transduction activity, lymphocyte activation or metabolism, and the immune response. Anti-SPEX antibodies are also useful as a detection agent in diagnostics and kits useful in detecting, purifying, and/or sorting SPEX expressing lymphocyte populations.

A preferred antibody is an anti-SPEX monoclonal antibody, preferably a human or humanized anti-SPEX monoclonal antibody. Other preferred anti-SPEX antibodies specifically immunoreact with one of a hSPEX polypeptide, a mSPEX polypeptide, or the mSPEXb polypeptide (the allele of murine SPEX disclosed above). An anti-SPEX antibody that immunoreacts with SEQ ID NO:7, SEQ ID NO:9, and/or SEQ ID NO: 11 is contemplated to immunoreact with all murine and human SPEX polypeptides including a SPEX tyrosine based domain (alternately with mammalian SPEX polypeptides which have a SPEX tyrosine based domain or an intracellular domain. Other exemplary anti-SPEX antibodies immunoreact with a SPEX polypeptide set forth in TABLE 1 and TABLE 2, TABLE 3, and TABLE 4. In certain embodiments, the anti-SPEX antibody immunoreacts with a first SPEX polypeptide, but does not immunoreact with a second SPEX polypeptide. For example, an anti-SPEX antibody immunoreacts with a SPEX polypeptide set forth in column 1 and/or column 2 of TABLE 1, but does not immunoreact with a SPEX polypeptide set forth in column 3 of TABLE 1. In another example, an anti-SPEX antibody immunoreacts with a SPEX polypeptide set forth in column 1 of TABLE 2, but does not immunoreact with a second SPEX polypeptide set forth in column 2 of TABLE 2.

The antigenic immunoreactivities of certain preferred anti-SPEX antibodies along with exemplary sequences, set forth by sequence identifier, are provided in TABLE 8, below.

TABLE 8
PREFERRED ANTI-SPEX ANTIBODIES IMMUNOREACT
WITH THE FOLLOWING ANTIGENS

| | Exemplary hSPEX | Exemplary mSPEX | |
|---|---|---|---|
| | Polypeptide | Polypeptide | Optionally not |
| | Antigen(s) | Antigen(s) | Immunoreactive with |
| SPEX Antigen | (SEQ ID NO) | (SEQ ID NO) | (SEQ ID NO) |
| Extracellular Domain | 12, 13 | 54, 55, 85, 86 | 16, 17, 18, 58, 59, 60 |
| Extracellular/Transmembrane | 14, 15 | 56, 57 | 16, 17, 58, 59 |
| Ig Like Domain | 3 | 45, 88 | 16, 17, 18, 58, 59, 60 |
| SPEX | 20, 21 | 62, 63 | |
| Exemplary SPEX Fusion | 98 | 99, 100 | 16, 17, 18, 58, 59, 60 |
| Polypeptide | | | |
| Intracellular Domain | 7, 9, 11, 16, 17, 18 | 7, 9, 11, 58, 59, 60 | 3, 45, 12, 13, 54, 55 |
| Intracellular/Transmembrane | 18 | 60 | 3, 45, 12, 13, 54, 55, |
| | | | 14, 15, 56, 57 |

One embodiment provides an antibody, preferably monoclonal, that immunoreacts with an extracellular domain of a SPEX polypeptide and is capable of modulating the proliferation or other metabolism of lymphocytes. More preferably, the antibody comprises a humanized monoclonal antibody. In one embodiment, administration of an antibody that immunoreacts with a SPEX extracellular domain, preferably the Ig like domain, to lymphocytes including an expressed SPEX polypeptide; inhibits proliferation and/or differentiation of the lymphocyte.

Optionally an anti-SPEX antibody may include a binding domain for a second epitope, for instance a different SPEX epitope or even a non-SPEX epitope (e.g., a multivalent antibody capable of binding one or more different epitopes).

In one embodiment, an anti-SPEX antibody immunoreacts with a human SPEX polypeptide, but does not substantially immunoreact with a mouse SPEX polypeptide. Optionally, an anti-SPEX antibody immunoreacts with a mouse SPEX polypeptide, but does not substantially immunoreact with a human SPEX polypeptide. Such antibodies are useful, for example, to distinguish human and mouse SPEX polypeptide sequences.

Methods of manufacturing an antibody given a specific antigen are well known in the art. The present invention provides SPEX antigens (i.e., immunogen) useful for manufacture of anti-SPEX antibodies. Thus, in light of the present disclosure, one of ordinary skill in the art is able to manufacture an anti-SPEX antibody of the present invention. Antibodies are commonly manufactured, for example: in animals (e.g., rabbit, mouse, hamster, sheep, goat, horse, bovine); in cells, primarily cell culture, (e.g., bacteria, plant, algae, insect, mammalian, murine, hybridoma, and human cells); by phage display; and by epitope cloning into antibody scaffold vectors and gene transfer into any of a variety of cell types (e.g., bacteria, plant, algae, insect, mammalian, murine, and human). Accordingly, the present invention also provides a cell capable of producing an anti-SPEX antibody.

In certain embodiments, the anti-SPEX antibody includes a detectable tag. Examples of a detectable tag include a radioisotope (e.g., ¹²⁵I), a fluorescent molecule (e.g., SMCC-activated BSA-Fluorescein, Prozyme, San Leandro, Calif.), biotinylation, myc tag, FLAG tag, and enzymes (e.g., peroxidase). Detectable tags and the labeling or conjugation of an antibody with a desirable detectable tag is well known in the art. Typically, the detectable tag is combined with the antibody by a covalent bond. Numerous commercial products are available for detectably labeling an antibody. It is preferred that the label does not interfere with structure and/or function of the variable portion of the antibody. Labeling in alternative areas of an antibody molecule or active fragment thereof are well known in the art.

In certain embodiments, it is desirable that an anti-SPEX antibody immunoreacts with a first SPEX polypeptide, but does not immunoreact with a second SPEX polypeptide. Monoclonal and polyclonal anti-SPEX antibodies having the present property can be prepared, for example, using epitope specific SPEX antigens in the manufacture of the anti-SPEX antibody. If desired, an antibody population can be screened to subtract or otherwise remove molecules in the population having an undesirable immunoreactivity (i.e., by subtraction screening on a matrix containing the undesirable antigen).

5. Method of Screening for SPEX Binding Partners

One embodiment provides a method of screening candidate molecules to identify one or more SPEX binding partners, comprising: 1) contacting a candidate molecule with a SPEX polypeptide; 2) determining whether or not the candidate molecule and the SPEX polypeptide form a binding complex, wherein the formation of a binding complex indicates that the candidate molecule is a SPEX binding partner; and 3) repeating steps 1) and 2) until the SPEX binding partner is identified. Preferred candidate binding partner molecules include small molecule organic compounds and polypeptides. Preferred SPEX polypeptide binding targets include a SPEX tyrosine based domain (e.g., SEQ ID NO:7, SEQ ID NO:9, or SEQ ID NO:11) or a SPEX Ig like domain (e.g., SEQ ID NO:3, SEQ ID NO:45, or SEQ ID NO:88).

In light of the present invention and the knowledge in the art, the determination of whether or not the candidate molecule and the SPEX polypeptide form a binding complex can be made using a variety of binding assays well known in the art, so the invention is not limited by the particular binding assay. For example, the binding complex can be detected by a molecular weight increase compared to the SPEX polypeptide alone with capture of the binding complex using an anti-SPEX antibody. The SPEX polypeptide may be engineered to include an affinity tag or a detectable label such that the binding complex can be identified through a change in molecular weight or migration through a gel, or direct detection of the SPEX polypeptide in a complex. Certain candidate molecules may have a known detectable property, such as an antibody immunoreactive site (epitope), fluorescent property, or affinity tag (natural or engineered) such that the candidate molecule can be directly detected in a binding complex with a SPEX polypeptide.

6. Method of Isolating a SPEX Polypeptide

One embodiment provides a method of purifying a SPEX polypeptide from a biological sample containing the SPEX polypeptide, comprising: contacting the biological sample with an affinity matrix having an anti-SPEX antibody attached to the matrix to produce an immunocomplex including the SPEX polypeptide and the antibody attached to the matrix; separating the remainder of the biological sample from the matrix; separating the SPEX polypeptide from the anti-SPEX antibody; and collecting the SPEX polypeptide, thereby obtaining the purified SPEX polypeptide.

7. Method of Regulating an Activity of Lymphocytes

One embodiment provides a method of modulating a metabolism (preferably proliferation or activation) of a lymphocyte that expresses a SPEX receptor, comprising contacting the lymphocyte with an anti-SPEX antibody (preferably a monoclonal antibody) that immunoreacts with an extracellular domain of the SPEX polypeptide. Certain embodiments disclose that SPEX receptor activity modulates, preferably inhibits metabolism, and more preferably inhibits proliferation, of lymphocytes that express the SPEX receptor. An antibody that immunoreacts with the extracellular domain of the SPEX polypeptide, preferably the Ig like domain, is disclosed in certain embodiments herein to inhibit SPEX activity providing a release from SPEX induced suppression of the metabolism thus the antibody provides a corresponding increase in the metabolism of the SPEX expressing lymphocyte. Useful antibodies include the rat anti-SPEX antibodies PK3, PK18, and PK23 (or immunoreactive fragments thereof). Preferred antibodies include humanized or human antibodies that immunoreact with an extracellular domain of a SPEX polypeptide. Preferred lymphocytes include B cells and T cells. In certain preferred embodiments, the antibody immunoreacts with an amino acid sequence set forth in SEQ ID NO:13, SEQ ID NO:55, or SEQ ID NO:85. The lymphocytes can be in an in vitro culture or in vivo. Lymphocytes are contacted with the antibody in vivo, for example, by administering the antibody or immunoreactive fragment thereof (e.g., by injection) to a human, or non-human mammal.

### EXAMPLES

### 1. Identify and Clone mSPEX cDNA

Positive selection is a developmental process in which immature thymocytes receive maturation signals as a consequence of T cell antigen receptor (TCR) recognition of MHC/self-peptide complexes expressed by thymic stroma. Exposure of immature thymocytes to low concentrations of the pharmacologic activators phorbol ester (e.g., PMA) and/or ionomycin induces the survival and differentiation of double positive (DP) thymocytes in vitro and provides an accepted model system of positive selection of thymocytes in vivo.

Using nucleic acid microarrays, the inventors identified changes in nucleic acid expression during positive selection of thymocytes relative to unstimulated thymocytes. TCRα-chain deficient thymocytes (murine) were cultured in medium in the presence or absence of 0.2 ng/ml PMA and 0.2 mg/ml ionomycin providing activated and non-activated thymocytes respectively. TCRα-chain deficient thymocytes are blocked at the DP stage of development due to a genetic mutation. Gene expression in these cells, after stimulation, is characteristic of developing thymocytes. After incubating the cells for 6 hours, the poly-A+ RNA was isolated using RNeasy RNA and Oligotex mRNA kits (Qiagen). The poly-A+ RNA was subjected to comparative cDNA array analysis using the Mouse 1.02 Array per manufacturer's instructions (Incyte Genomics). Signal analysis of the microarrays was performed using GEMTools analysis software (Incyte Genomics).

The inventors selected two sequences for further study based upon the ratio of expression of the sequences in the stimulated thymocytes over the unstimulated thymocytes. The ratio of expression was determined from the normalized signals of hybridization of each labeled cDNA to the ESTs AA184189 and AA177302, respectively, which ESTs were included on the Mouse Array. The expression of each sequence in the stimulated thymocytes was increased 9.1 fold and 6.6 fold for sequences hybridizing to EST AA184189 and EST AA177302, respectively. These ESTs on the Mouse 1.02 Array originated from a murine library called the Soares mouse 3NbMS library which in turn was derived from spleens of 4 week old C57BL/6J mice.

The inventors determined that the ESTs AA184189 and AA177302 are linked using BLAST software (NCBI) to compare the sequences of the ESTs with a database of murine nucleic acid sequences (each EST corresponds to a common sequence in the murine sequence database). Thus, ESTs AA184189 and AA177302 are determined to be part of a more complete nucleic acid. Using the 5′-most EST (AA177302), the inventors cloned a full-length gene by rapid amplification of cDNA ends (SMART RACE cDNA amplification kit, Clontech) using cDNA prepared from stimulated thymocytes as a template in the reaction.

The present gene is designated herein as the mouse spleen expressed gene (mSPEX gene). An exemplary mSPEX nucleic acid is set forth in SEQ ID NO:105 and includes a coding region set forth in SEQ ID NO:84. A BLAST search of GenBank reveals that the SPEX cDNA encodes a novel protein. An exemplary mSPEX polypeptide encoded by an mSPEX nucleic acid is set forth in SEQ ID NO:63. A signal sequence (e.g., SEQ ID NO:43) is typically cleaved during cellular processing to form an exemplary mature mSPEX polypeptide set forth in SEQ ID NO:62. Using sequence comparison between the mSPEX polypeptide and the Pfam family of proteins database (NCBI), the inventors identified an immunoglobulin like domain in a predicted extracellular portion of the polypeptide; however, the inventors observed that SPEX lacks close homology to any particular immunoglobulin containing superfamily member.

### 2. Identify and Clone hSPEX cDNA

Using sequence comparisons of the mSPEX sequence to human sequences in the GenBank database the inventors determine that the human ESTs AI792952 and AA931122 align with the mSPEX polynucleotide and that the human ESTs correspond to a single human clone in the GenBank database. A bacterial stab (AI792952) containing the human clone, as well as numerous contaminating clones, was purchased from the IMAGE Consortium (Lawrence Livermore National Laboratory, Livermore, Calif.). A human gene sequence homologous to mSPEX is purified from the stab, subcloned, and sequenced. An exemplary hSPEX nucleic acid sequence is set forth in SEQ ID NO: 104 and includes a coding region set forth in SEQ ID NO:42. An exemplary hSPEX polypeptide encoded by an hSPEX nucleic acid sequence is set forth in SEQ ID NO:21. A signal sequence (e.g., SEQ ID NO: 1) is typically cleaved during cellular processing of hSPEX protein to form an exemplary mature hSPEX polypeptide set forth in SEQ ID NO:20.

### 3. Anti-mSPEX Monoclonal Antibody

An isolated expression vector construct (referred to herein as p-mSPEX-Ig) comprising a coding region encoding a fusion protein, the fusion having a mSPEX extracellular domain (including the SPEX signal sequence) and the hinge, CH2, and CH3 domains of human IgG1 is prepared. The p-mSPEX-Ig vector is transfected into 293 cells in culture. The p-mSPEX-Ig transfected 293 cells express and secrete the mSPEX-Ig fusion protein as assessed by a Western blot probed with ani-humanIgG1 antibody. The mSPEX-Ig fusion protein is purified by protein A affinity chromatography from supernatants of transfected 293 cells.

Rats are immunized in the base of the tail with the purified recombinant mSPEX-Ig emulsified in CFA to produce monoclonal antibodies. Procedures and techniques for the production of antibodies, including monoclonal antibodies, to a given antigen are well known in the art. The medial iliac lymph nodes are harvested two weeks later and fused with YB2/0 cells by standard methods. Antibodies thus produced are screened for specific immunoreactivity with mSPEX by FACS using DPK and 293 cells that are transfected to express a cell surface mSPEX-YFP fusion protein (YFP is an abbreviation for yellow fluorescent protein). Screening continues until one or more anti-mSPEX monoclonal antibody is identified in the screening process. The YFP tag allows verification that transfected cells express the mSPEX-YFP fusion protein at the cell surface and allows the correlation of the relative level of cellular antibody binding to mSPEX-YFP fusion protein expression. Specifically, the DPK and 293 cells used for the screening procedure are transfected with an isolated expression vector construct made from cloning a mSPEX gene insert into the pEYEP-N1 vector (Clontech). The pEYEP-N1 vector supplies an YFP tag and expression of a mSPEX insert from pEYEP-N1 results in the expression of a fusion protein including a mSPEX polypeptide operably linked with a YFP tag. Three hybridomas PK3, PK18, and PK23 are obtained from this screen. The hybridomas are optionally re-cloned and antibody is purified using standard methods.

The monoclonal antibody thus produced specifically immunoreacts with the extracellular domain of mSPEX. Three monoclonal antibodies that immunoreact with the extracellular domain of mSPEX are: PK3, PK18, and PK23; as referred to herein.

### 4. Anti-hSPEX Monoclonal Antibody

A monoclonal antibody that specifically reacts with the extracellular domain of a hSPEX polypeptide is prepared using the methods disclosed in Example 3, except that 1) a p-hSPEX-Ig expression vector comprising a coding region encoding a fusion protein, the fusion having a mSPEX extracellular domain (including the SPEX signal sequence) and the hinge, CH2, and CH3 domains of human IgG1 is prepared and used to produce monoclonal antibodies in rats and 2) a hSPEX polynucleotide encoding the extracellular domain of a hSPEX polypeptide is cloned into a pEYEP-N1 vector and expressed in DPK and 293 cells to screen for monoclonal antibodies using the FACS-YFP procedure disclosed above. The monoclonal antibody produced specifically immunoreacts with the extracellular domain of hSPEX.

### 5. Map Kinase Signaling Stimulates SPEX Expression

The expression of mSPEX and hSPEX mRNAs is compared in cultured DP thymocytes in the presence or absence of stimulation with PMA and ionomycin and in the presence or absence of a MEK inhibitor (10 μM U0126). The increase of SPEX mRNA expression (in both murine and human cells) observed with PMA and ionomycin stimulation is inhibited by the MEK inhibitor by approximately 70% in one experiment. These data are consistent with MAP kinase signaling leading to stimulation of SPEX mRNA expression.

### 6. Isolation of Cellular mSPEX

The mSPEX protein is isolated under reducing and non-reducing conditions from murine splenocyte whole cell lysates by affinity chromatography using a matrix bound anti-mSPEX monoclonal antibody. The matrix bound antibody is contacted with the cell lysates under conditions sufficient for binding of mSPEX polypeptide in the lysates with the antibody thereby forming a reaction complex including the matrix, the anti-hSPEX antibody, and hSPEX polypeptide (immunoreaction buffers and other conditions, such as incubation time and temperature, are well known in the art and are applicable here). The matrix is washed to remove non-mSPEX contaminants. The mSPEX polypeptide is released from the matrix-antibody-mSPEX complex and the mSPEX polypeptide is collected in purified form. The presence of purified mSPEX polypeptide is confirmed by electrophoresis on SDS-PAGE gels using silver staining to detect biological contents of the samples. The mSPEX polypeptide is essentially the only material present in the samples, appearing as a 35 kDa and 37 kDa doublet as detected by silver staining. The identity of each band of the doublet is confirmed to be mSPEX by epitope tagging of transfected cells and by Western blotting using an anti-mSPEX monoclonal antibody.

### 7. Isolation of Cellular hSPEX

The hSPEX protein is isolated under reducing and non-reducing conditions from human splenocytes cell culture lysates by affinity chromatography using a matrix bound anti-hSPEX monoclonal antibody. The matrix bound antibody is contacted with the cell lysates under conditions sufficient for binding of hSPEX polypeptide in the lysates with the antibody thereby forming a reaction complex including the matrix, the anti-hSPEX antibody, and hSPEX polypeptide. The matrix is washed to remove non-hSPEX contaminants. The hSPEX polypeptide is released from the matrix-antibody-hSPEX complex and the hSPEX polypeptide is collected in purified form. The presence of purified hSPEX polypeptide in the sample is confirmed by electrophoresis on SDS-PAGE gels using silver staining to detect the contents of the sample. The hSPEX polypeptide is essentially the only material present in the samples as detected by silver staining. The identity of the hSPEX polypeptide is confirmed by Western blotting using an anti-hSPEX monoclonal antibody.

### 8. Tissue Distribution of SPEX Protein

The tissue distribution of SPEX gene expression is examined in murine brain, heart, kidney, liver, lung, muscle, skin, small intestine, spleen, stomach, testis, and thymus tissues by Northern blot analysis of cellular mRNA. Essentially no SPEX mRNA is detected by Northern analysis in murine brain or testis. Low level expression of SPEX is observed in heart, kidney, muscle, and skin tissues. Intermediate level expression of SPEX is observed in liver, lung, small intestine, and stomach tissues. High level expression of SPEX is observed in the thymus and spleen tissues with about 2 fold greater expression in the spleen tissue compared to the thymus tissue.

The distribution of SPEX mRNA expression in lymphocytes is evaluated in murine B-lymphocytes, CD4+ thymocytes, and CD8+ thymocytes. Expression of SPEX in thymocytes is determined by 3 color staining the thymocytes for CD4, CD8, and SPEX and analyzing the expression patterns by flow cytometry. The SPEX mRNA expression is higher on CD4 T cells compared to CD8 T cells. In one experiment the CD4 T cell SPEX mRNA expression over CD8 T cell SPEX mRNA expression was approximately 2:1. The expression of SPEX protein is also higher on CD4 T cells compared to the CD8 lineage, although both population of cells express the protein. These data are consistent with SPEX signaling participating in lineage commitment of T cells.

Expression of SPEX mRNA in B-cells, CD4 T-cells, and CD8 T-cells is evaluated by 3 color staining murine spleen cells and analyzing the expression patterns by FACS. Again, SPEX is expressed on B and T cells with an typical order of expression level from highest to lowest being B cells, followed by CD4 T cells, and then by CD8 cells.

### 9. Regulation of T Cell Response With Anti-SPEX Antibody

This example detects the modulation of a T cell metabolism by contacting a T cell with an anti-mSPEX antibody. CD4 T cells are purified by magnetic bead depletion from lymph nodes of AND TCR transgenic mice. These T cells have a well characterized antigen specificity regarding CD4 and CD8 expression. In the present assay, the CD4 T cells are admixed with antigen presenting cells (APCs) and an antigen in the presence or absence of a SPEX specific monoclonal antibody. Cultures of each admixture are incubated for different selected time periods and the T cell response is detected or measured. A change in the T cell proliferation rate in the antibody treated admixtures compared to admixtures without the antibody is evidence of modulation of T cell metabolism resulting from the administration of the antibody which immunoreacts with an extracellular domain of SPEX.

The APCs in the above assay are prepared from irradiated spleen cells of BALB.K mice according to standard techniques known in the art. The APCs have the appropriate MHC molecules to present antigen to the AND T cells, have the mSPEXb allele, and express the mSPEXb polypeptide; but do not have the SPEX allele or express a mSPEX polypeptide other than mSPEXb. The antibody used is PK18 which is an anti-mSPEX antibody which does not immunoreact with the mSPEXb polypeptide. The PK18 antibody immunoreacts with the extracellular domain of the mSPEX polypeptide. Thus, the APCs prepared from BALB.K mice do not immunoreact with the PK18 antibody. T cells and APC are mixed in the presence of various concentrations of antigen, in this example a peptide derived from pigeon cytochrome c, in the presence or absence of PK18 anti-SPEX monoclonal antibody, and cultured for various time periods. Proliferation is measured by ³H thymidine uptake at various time periods after the start of culture.

### 10. Inhibition of T Cell Activation With an Anti-SPEX Antibody

This example demonstrates a method of inhibiting T cell activation by contacting a T cell containing a SPEX polypeptide with an antibody that immunoreacts with the SPEX polypeptide. LN T cells are separately purified from B10.BR (which have a mSPEX polypeptide) and BALB.K mice (which have a mSPEXb polypeptide). The T cells are cultured in the presence of 3H-TdR (tritiated thymidine) in 96-well plates coated with equal amounts of anti-CD3 and rat Ig or equal amounts of anti-CD3 and monoclonal antibody PK18. The anti-CD3 antibody is a known stimulator of T cell activation. The PK18 antibody immunoreacts with the mSPEX polypeptide of the B10.BR T cells, but does not immunoreact with the mSPEXb polypeptide of the BALB.K T cells.

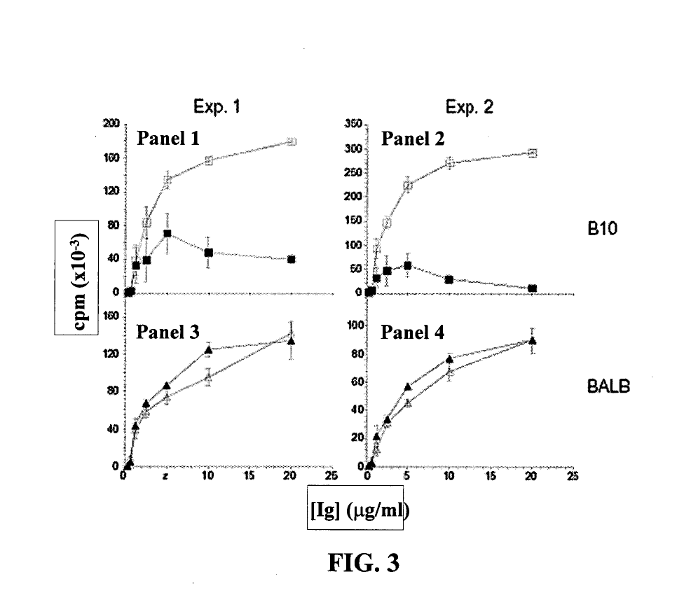

Results are shown in FIG. 3. 3H-TdR incorporation into T cells reacted with anti-CD3 and rat Ig is depicted as open symbols in FIG. 3. 3H-TdR incorporation into T cells reacted with anti-CD3 and PK18 is depicted as closed symbols in FIG. 3. The data are expressed as the mean 3H-TdR incorporation for triplicate cultures (+/− standard deviation (SD)) minus the cpm of 3H-TdR incorporation of cultures of T cells alone (which was less than 1500 cpm). The incorporation of 3H-TdR into the T cells is correlated with both proliferation of the T cells and with T cell activation.

Panels 1 and 2 (two different experiments) of FIG. 3 demonstrate that the activation of T cells from B10 mice is inhibited when the T cells are contacted with the antibody PK18 which immunoreacts with the mSPEX of the B10 mouse T cells. Panels 3 and 4 (two different experiments) of FIG. 3 demonstrate that the activation of T cells from BALB.K mice is not significantly modulated when the T cells are contacted with the antibody PK18 which does not immunoreact with the mSPEXb of the BALB.K mouse T cells.

The PK18 antibody immunoreacts with the extracellular domain of the mSPEXb polypeptide. Thus, the data depicted in FIG. 3 demonstrate a method of inhibiting T cell activation by contacting a T cell containing a SPEX polypeptide with an antibody that immunoreacts with the SPEX polypeptide, preferably the extracellular domain of the SPEX polypeptide, and more preferably the Ig like domain of the SPEX polypeptide.

### 11. Inhibition of B Cell Activation With an Anti-SPEX Antibody

This example demonstrates a method of inhibiting B cell activation by contacting a B cell containing a SPEX polypeptide with an antibody that immunoreacts with the SPEX polypeptide. Example 10 is repeated except that B cells are purified from the B10.BR and BALB.K mice. It is found that B cell activation is inhibited by contacting the B cells of the B10, but not the BALB mice with the PK18 antibody. Thus, the data demonstrate a method of inhibiting B cell activation by contacting a B cell containing a SPEX polypeptide with an antibody that immunoreacts with the SPEX polypeptide, preferably the extracellular domain of the SPEX polypeptide, and more preferably the Ig like domain of the SPEX polypeptide.

### 12. Murine Anti-MSPEX Monoclonal Antibody

BALB/c mice are immunized (50% g in CFA) and boosted (3 to 5 times, 50% g in IFA) with recombinant soluble SPEX-Ig fusion protein (including the extracellular domain of SPEX as discussed above). The immunogen is derived from B6 mice (containing the mSPEX allele, not the mSPEXb allele) and thus generates allele specific antibodies in BALB/c mice. Hybridomas are produced by standard means from splenocytes of immunized animals. Monoclonal antibodies are screened by differential binding to parental and transfected cells expressing SPEX-YFP fusion protein. Three monoclonal antibodies, PJ16, PJ19 and PJ196, are selected for additional analysis. The isotype of each antibody is IgG1κ. The PJ19, PJ16, and PJ196 antibodies have identical staining patterns to the PK series of rat monoclonal antibodies (see above), including specifically immunoreacting with mSPEX (but not mSPEXb) expressed by B6 cells, but not BALB/c derived cells (which express mSPEXb, but not mSPEX).

Various modifications of the invention, in addition to the embodiments provided herein, will become apparent to those skilled in the art from the foregoing description and fall within the scope of the claims.

What is claimed is:

1) A purified polypeptide comprising an amino acid sequence set forth in SEQ ID NO:3.

2) The polypeptide of claim 1, wherein the amino acid sequence comprises an immunoglobulin like domain.

3) The polypeptide of claim 1, wherein the polypeptide does not include a transmembrane domain.

4) The polypeptide of claim 1, wherein the polypeptide does not include SEQ ID NO:5.

5) The polypeptide of claim 1, wherein the polypeptide does not include a tyrosine based domain.

6) The polypeptide of claim 1, wherein the polypeptide does not include SEQ ID NO:7.

7) The polypeptide of claim 1, wherein the polypeptide does not include SEQ ID NO:9.

8) The polypeptide of claim 1, wherein the polypeptide does not include SEQ ID NO: 11.

9) The polypeptide of claim 1, wherein the polypeptide includes SEQ ID NO: 12 or SEQ ID NO: 13.

10) The polypeptide of claim 1, wherein the polypeptide includes a transmembrane domain.

11) The polypeptide of claim 1, wherein the polypeptide includes SEQ ID NO:14 or SEQ ID NO:15.

12) The polypeptide of claim 1, wherein the polypeptide includes a tyrosine based domain.

13) The polypeptide of claim 1, wherein the polypeptide includes SEQ ID NO: 19, SEQ ID NO:20, or SEQ ID NO:21.

14) The polypeptide of claim 1, further comprising a second amino acid sequence operably linked to the first amino acid sequence.

15) The polypeptide of claim 1, further comprising a second amino acid sequence, wherein the second sequence is more soluble in aqueous solution than the first amino acid sequence.

16) The polypeptide of claim 1, further comprising a detectable marker.

17) The polypeptide of claim 1, further comprising an affinity tag.

18) The polypeptide of claim 1, further comprising a second amino acid sequence set forth in SEQ ID NO:97.

19) A purified polypeptide comprising an amino acid sequence set forth in SEQ ID NO:45.

20) The polypeptide of claim 19, wherein the amino acid sequence comprises an immunoglobulin like domain.

21) The polypeptide of claim 19, wherein the polypeptide does not include a transmembrane domain.

22) The polypeptide of claim 19, wherein the polypeptide does not include SEQ ID NO:47.

23) The polypeptide of claim 19, wherein the polypeptide does not include a tyrosine based domain.

24) The polypeptide of claim 19, wherein the polypeptide does not include SEQ ID NO:7, SEQ ID NO:9, or SEQ ID NO:11.

25) The polypeptide of claim 19, wherein the polypeptide includes SEQ ID NO:54 or SEQ ID NO:55.

26) The polypeptide of claim 19, wherein the polypeptide includes a transmembrane domain.

27) The polypeptide of claim 19, wherein the polypeptide includes SEQ ID NO:56 or SEQ ID NO:57.

28) The polypeptide of claim 19, wherein the polypeptide includes a tyrosine based domain.

29) The polypeptide of claim 19, wherein the polypeptide includes SEQ ID NO:61, SEQ ID NO:62, or SEQ ID NO:63.

30) The polypeptide of claim 19, further comprising a second amino acid sequence operably linked to the first amino acid sequence.

31) The polypeptide of claim 19, further comprising a second amino acid sequence, wherein the second sequence is more soluble in aqueous solution than the first amino acid sequence.

32) The polypeptide of claim 19, further comprising a detectable marker.

33) The polypeptide of claim 19, further comprising an affinity tag.

34) The polypeptide of claim 19, further comprising a second amino acid sequence set forth in SEQ ID NO:97.

35) A purified polypeptide comprising an amino acid sequence set forth in SEQ ID NO:88.

36) The polypeptide of claim 35, wherein the amino acid sequence comprises an immunoglobulin like domain.

37) The polypeptide of claim 35, wherein the polypeptide does not include a transmembrane domain.

38) The polypeptide of claim 35, wherein the polypeptide does not include a tyrosine based domain.

39) The polypeptide of claim 35, wherein the polypeptide does not include SEQ ID NO:7, SEQ ID NO:9, or SEQ ID NO: 11.

40) The polypeptide of claim 35, wherein the polypeptide includes a transmembrane domain.

41) The polypeptide of claim 35, wherein the polypeptide includes a tyrosine based domain.

42) The polypeptide of claim 35, further comprising a second amino acid sequence operably linked to the first amino acid sequence.

43) The polypeptide of claim 35, further comprising a second amino acid sequence, wherein the second sequence is more soluble in aqueous solution than the first amino acid sequence.

44) The polypeptide of claim 35, further comprising a detectable marker.

45) The polypeptide of claim 35, further comprising an affinity tag.

46) The polypeptide of claim 35, further comprising a second amino acid sequence set forth in SEQ ID NO:97.

47) A purified polypeptide, comprising an amino acid sequence set forth in SEQ ID NO:7.

48) The polypeptide of claim 47, wherein the amino acid sequence comprises a tyrosine based domain.

49) The polypeptide of claim 47, wherein the polypeptide further includes SEQ ID NO:9.

50) The polypeptide of claim 47, wherein the polypeptide further includes SEQ ID NO: 11.

51) The polypeptide of claim 47, wherein the polypeptide further includes SEQ ID NO:9 and SEQ ID NO: 11.

52) The polypeptide of claim 47, wherein the polypeptide does not include a transmembrane domain.

53) The polypeptide of claim 47, wherein the polypeptide does not include SEQ ID NO:5 or SEQ ID NO:47.

54) The polypeptide of claim 47, wherein the polypeptide does not include an immunoglobulin like domain.

55) The polypeptide of claim 47, wherein the polypeptide does not include SEQ ID NO:3, SEQ ID NO:45, or SEQ ID NO:88.

56) The polypeptide of claim 47, wherein the polypeptide includes SEQ ID NO: 16.

57) The polypeptide of claim 47, wherein the polypeptide includes SEQ ID NO: 17.

58) The polypeptide of claim 47, wherein the polypeptide includes SEQ ID NO: 18.

59) The polypeptide of claim 47, wherein the polypeptide includes SEQ ID NO:58.

60) The polypeptide of claim 47, wherein the polypeptide includes SEQ ID NO:59.

61) The polypeptide of claim 47, wherein the polypeptide includes SEQ ID NO:60.

62) The polypeptide of claim 47, wherein the tyrosine based domain comprises a phosphorylation site.

63) The polypeptide of claim 47, further comprising a second amino acid sequence operably linked to the first amino acid sequence.

64) The polypeptide of claim 47, further comprising a detectable marker.

65) The polypeptide of claim 47, further comprising an affinity tag.

66) A purified polypeptide, comprising an amino acid sequence set forth in SEQ ID NO:9.

67) The polypeptide of claim 66, wherein the amino acid sequence comprises a tyrosine based domain.

68) The polypeptide of claim 66, wherein the polypeptide further includes SEQ ID NO:7.

69) The polypeptide of claim 66, wherein the polypeptide further includes SEQ ID NO: 11.

70) The polypeptide of claim 66, wherein the polypeptide further includes SEQ ID NO:7 and SEQ ID NO: 11.

71) The polypeptide of claim 66, wherein the polypeptide does not include a transmembrane domain.

72) The polypeptide of claim 66, wherein the polypeptide does not include SEQ ID NO:5 or SEQ ID NO:47.

73) The polypeptide of claim 66, wherein the polypeptide does not include an immunoglobulin like domain.

74) The polypeptide of claim 66, wherein the polypeptide does not include SEQ ID NO:3, SEQ ID NO:45, or SEQ ID NO:88.

75) The polypeptide of claim 66, wherein the polypeptide includes SEQ ID NO:16.

76) The polypeptide of claim 66, wherein the polypeptide includes SEQ ID NO: 17.

77) The polypeptide of claim 66, wherein the polypeptide includes SEQ ID NO: 18.

78) The polypeptide of claim 66, wherein the polypeptide includes SEQ ID NO:58.

79) The polypeptide of claim 66, wherein the polypeptide includes SEQ ID NO:59.

80) The polypeptide of claim 66, wherein the polypeptide includes SEQ ID NO:60.

81) The polypeptide of claim 66, wherein the tyrosine based domain comprises a phosphorylation site.

82) The polypeptide of claim 66, further comprising a second amino acid sequence operably linked to the first amino acid sequence.

83) The polypeptide of claim 66, further comprising a detectable marker.

84) The polypeptide of claim 66, further comprising an affinity tag.

85) A purified polypeptide, comprising an amino acid sequence set forth in SEQ ID NO: 11.

86) The polypeptide of claim 85, wherein the amino acid sequence comprises a tyrosine based domain.

87) The polypeptide of claim 85, wherein the polypeptide further includes SEQ ID NO:7.

88) The polypeptide of claim 85, wherein the polypeptide further includes SEQ ID NO:9.

89) The polypeptide of claim 85, wherein the polypeptide further includes SEQ ID NO:7 and SEQ ID NO:9.

90) The polypeptide of claim 85, wherein the polypeptide does not include a transmembrane domain.

91) The polypeptide of claim 85, wherein the polypeptide does not include SEQ ID NO:5.

92) The polypeptide of claim 85, wherein the polypeptide does not include SEQ ID NO:47.

93) The polypeptide of claim 85, wherein the polypeptide does not include an immunoglobulin like domain.

94) The polypeptide of claim 85, wherein the polypeptide does not include SEQ ID NO:3 or SEQ ID NO:45.

95) The polypeptide of claim 85, wherein the polypeptide includes SEQ ID NO: 16.

96) The polypeptide of claim 85, wherein the polypeptide includes SEQ ID NO: 17.

97) The polypeptide of claim 85, wherein the polypeptide includes SEQ ID NO: 18.

98) The polypeptide of claim 85, wherein the polypeptide includes SEQ ID NO:58.

99) The polypeptide of claim 85, wherein the polypeptide includes SEQ ID NO:59.

100) The polypeptide of claim 85, wherein the polypeptide includes SEQ ID NO:60.

101) The polypeptide of claim 85, wherein the tyrosine based domain comprises a phosphorylation site.

102) The polypeptide of claim 85, further comprising a second amino acid sequence operably linked to the first amino acid sequence.

103) The polypeptide of claim 85, further comprising a detectable marker.

104) The polypeptide of claim 85, further comprising an affinity tag.

105) A purified polypeptide comprising an amino acid sequence that is 95% or more identical to SEQ ID NO:3 and includes an immunoglobulin like domain structure.

106) The polypeptide of claim 105, wherein the immunoglobulin like domain structure is substantially similar to the structure of SEQ ID NO:3.

107) The polypeptide of claim 105, wherein the immunoglobulin like domain structure is substantially similar to the structure of SEQ ID NO: 12.

108) The polypeptide of claim 105, wherein the amino acid sequence is 98% or more identical to SEQ ID NO:3.

109) The polypeptide of claim 105, wherein the polypeptide does not include SEQ ID NO:5.

110) The polypeptide of claim 105 wherein the polypeptide does not include SEQ ID NO:7, SEQ ID NO:9, or SEQ ID NO:11.

111) The polypeptide of claim 105, wherein the polypeptide further includes SEQ ID NO:7, SEQ ID NO:9, or SEQ ID NO:11.

112) The polypeptide of claim 105, further comprising a second amino acid sequence operably linked with the first amino acid sequence.

113) The polypeptide of claim 112, wherein the second amino acid sequence includes a segment that is 95% or more identical to SEQ ID NO:16.

114) The polypeptide of claim 113, wherein the segment includes a polymer set forth in SEQ ID NO:7, SEQ ID NO:9, or SEQ ID NO: 11.

115) The polypeptide of claim 105, wherein the polypeptide is operably linked to a detectable marker or an affinity tag.

116) A purified polypeptide comprising an amino acid sequence that is 95% or more identical to SEQ ID NO:45 and includes an immunoglobulin like domain structure.

117) The polypeptide of claim 116, wherein the immunoglobulin like domain structure is substantially similar to the structure of SEQ ID NO:45.

118) The polypeptide of claim 116, wherein the immunoglobulin like domain structure is substantially similar to the structure of SEQ ID NO:54.

119) The polypeptide of claim 116, wherein the amino acid sequence is 98% or more identical to SEQ ID NO:45.

120) The polypeptide of claim 116, wherein the polypeptide does not include SEQ ID NO:47.

121) The polypeptide of claim 116, wherein the polypeptide does not include SEQ ID NO:7, SEQ ID NO:9, or SEQ ID NO: 11.

122) The polypeptide of claim 116, wherein the polypeptide further includes SEQ ID NO:7, SEQ ID NO:9, or SEQ ID NO:11.

123) The polypeptide of claim 116, further comprising a second amino acid sequence operably linked with the first amino acid sequence.

124) The polypeptide of claim 123, wherein the second amino acid sequence includes a segment that is 95% or more identical to SEQ ID NO:58.

125) The polypeptide of claim 124, wherein the segment includes a polymer set forth in SEQ ID NO:7, SEQ ID NO:9, or SEQ ID NO: 11.

126) The polypeptide of claim 116, wherein the polypeptide is operably linked to a detectable marker or an affinity tag.

127) A purified polypeptide comprising an amino acid sequence that is 95% or more identical to SEQ ID NO:88 and includes an immunoglobulin like domain structure.

128) The polypeptide of claim 127, wherein the immunoglobulin like domain structure is substantially similar to the structure of SEQ ID NO:88.

129) The polypeptide of claim 127, wherein the immunoglobulin like domain structure is substantially similar to the structure of SEQ ID NO:86.

130) The polypeptide of claim 127, wherein the amino acid sequence is 98% or more identical to SEQ ID NO:88.

131) The polypeptide of claim 127, wherein the polypeptide further includes SEQ ID NO:7, SEQ ID NO:9, or SEQ ID NO: 11.

132) The polypeptide of claim 127, further comprising a second amino acid sequence operably linked with the first amino acid sequence.

133) The polypeptide of claim 132, wherein the second amino acid sequence comprises a detectable marker or an affinity tag.

134) The polypeptide of claim 127, wherein the polypeptide is operably linked to a detectable marker or an affinity tag.

135) A purified immunogenic polypeptide, comprising ten consecutive amino acid residues of SEQ ID NO:3.

136) A purified immunogenic polypeptide, consisting essentially of 8 to 40 consecutive amino acid residues of SEQ ID NO:3.

137) A purified polynucleotide, comprising a nucleic acid sequence encoding a SPEX polypeptide, or a complement of the nucleic acid sequence.

138) The polynucleotide of claim 137, wherein the polynucleotide includes no more than 30000 nucleotides or nucleotide base pairs.

139) The polynucleotide of claim 137, wherein the polynucleotide includes no more than 15000 nucleotides or nucleotide base pairs.

140) The polynucleotide of claim 137, wherein the polynucleotide includes no more than 7000 nucleotides or nucleotide base pairs.

141) The polynucleotide of claim 137, wherein the polynucleotide includes 800 to 5000 nucleotides or nucleotide base pairs.

142) The polynucleotide of claim 137, wherein the polynucleotide includes an intervening sequence.

143) The polynucleotide of claim 137, wherein the polypeptide does not include a transmembrane domain.

144) The polynucleotide of claim 137, wherein the polypeptide does not include SEQ ID NO:5.

145) The polynucleotide of claim 137, wherein the polypeptide does not include SEQ ID NO:7.

146) The polynucleotide of claim 137, wherein the polypeptide does not include SEQ ID NO:9.

147) The polynucleotide of claim 137, wherein the polypeptide does not include SEQ ID NO:11.

148) The polynucleotide of claim 137, wherein the polypeptide includes SEQ ID NO:3.

149) The polynucleotide of claim 137, wherein the polypeptide includes SEQ ID NO: 12.

150) The polynucleotide of claim 137, wherein the polypeptide includes SEQ ID NO: 13.

151) The polynucleotide of claim 137, wherein the polypeptide includes SEQ ID NO:14.

152) The polynucleotide of claim 137, wherein the polypeptide includes SEQ ID NO: 15.

153) The polynucleotide of claim 137, wherein the polypeptide includes SEQ ID NO:19.

154) The polynucleotide of claim 137, wherein the polypeptide includes SEQ ID NO:20.

155) The polynucleotide of claim 137, wherein the polypeptide includes SEQ ID NO:21.

156) A purified polynucleotide, comprising a nucleic acid sequence encoding a polypeptide sequence set forth in SEQ ID NO:45, or a complement of the nucleic acid sequence.

157) The polynucleotide of claim 156, wherein the polynucleotide includes no more than 30000 nucleotides or nucleotide base pairs.

158) The polynucleotide of claim 156, wherein the polynucleotide includes no more than 15000 nucleotides or nucleotide base pairs.

159) The polynucleotide of claim 156, wherein the polynucleotide includes no more than 7000 nucleotides or nucleotide base pairs.

160) The polynucleotide of claim 156, wherein the polynucleotide includes 800 to 5000 nucleotides or nucleotide base pairs.

161) The polynucleotide of claim 156, wherein the polynucleotide includes an intervening sequence.

162) The polynucleotide of claim 156, wherein the polypeptide does not include a transmembrane domain.

163) The polynucleotide of claim 156, wherein the polypeptide does not include SEQ ID NO:47.

164) The polynucleotide of claim 156, wherein the polypeptide does not include SEQ ID NO:7.

165) The polynucleotide of claim 156, wherein the polypeptide does not include SEQ ID NO:9.

166) The polynucleotide of claim 156, wherein the polypeptide does not include SEQ ID NO:11.

167) The polynucleotide of claim 156, wherein the polypeptide includes SEQ ID NO:54.

168) The polynucleotide of claim 156, wherein the polypeptide includes SEQ ID NO:55.

169) The polynucleotide of claim 156, wherein the polypeptide includes SEQ ID NO:56.

170) The polynucleotide of claim 156, wherein the polypeptide includes SEQ ID NO:57.

171) The polynucleotide of claim 156, wherein the polypeptide includes SEQ ID NO:61.

172) The polynucleotide of claim 156, wherein the polypeptide includes SEQ ID NO:62.

173) The polynucleotide of claim 156, wherein the polypeptide includes SEQ ID NO:63.

174) A purified polynucleotide, comprising a nucleic acid sequence encoding a polypeptide sequence set forth in SEQ ID NO:88, or a complement of the nucleic acid sequence.

175) The polynucleotide of claim 174, wherein the polynucleotide includes no more than 30000 nucleotides or nucleotide base pairs.

176) The polynucleotide of claim 174, wherein the polynucleotide includes no more than 15000 nucleotides or nucleotide base pairs.

177) The polynucleotide of claim 174, wherein the polynucleotide includes no more than 7000 nucleotides or nucleotide base pairs.

178) The polynucleotide of claim 174, wherein the polynucleotide includes 800 to 5000 nucleotides or nucleotide base pairs.

179) The polynucleotide of claim 174, wherein the polynucleotide includes an intervening sequence.

180) The polynucleotide of claim 174, wherein the polypeptide does not include a transmembrane domain.

181) The polynucleotide of claim 174, wherein the polypeptide does not include SEQ ID NO:7.

182) The polynucleotide of claim 174, wherein the polypeptide does not include SEQ ID NO:9.

183) The polynucleotide of claim 174, wherein the polypeptide does not include SEQ ID NO: 11.

184) The polynucleotide of claim 174, wherein the polypeptide includes SEQ ID NO:85.

185) The polynucleotide of claim 174, wherein the polypeptide includes SEQ ID NO:86.

186) A purified polynucleotide, comprising a nucleic acid sequence encoding a SPEX tyrosine based domain, or a complement of the nucleic acid sequence.

187) A purified polynucleotide, comprising:
a) a nucleic acid sequence encoding a SPEX polypeptide, or a complement thereof; and
b) a vector sequence operatively linked to the nucleic acid sequence.

188) The polynucleotide of claim 187, wherein the vector sequence comprises an expression vector having one or more control elements for producing an expression product of the nucleic acid sequence.

189) The polynucleotide of claim 188, wherein the expression vector comprises an in vitro expression system.

190) The polynucleotide of claim 188, wherein the expression vector comprises a cellular expression system.

191) The expression vector of 190, wherein the expression vector includes one or more control elements for expressing the product in a human cell.

192) The polynucleotide of claim 190, wherein the expression vector includes one or more control elements for expressing the product in one or more cell types selected from a group consisting of: a bacterial cell, a yeast cell, an insect cell, and a mammalian cell.

193) The polynucleotide of claim 192, wherein the product comprises the complement of the nucleic acid sequence.

194) The polynucleotide of claim 192, wherein the product comprises the SPEX polypeptide.

195) The polypeptide of claim 194, wherein the polypeptide includes SEQ ID NO:3.

196) The polypeptide of claim 195, wherein the polypeptide does not include a transmembrane domain.

197) The polypeptide of claim 194, wherein the polypeptide includes SEQ ID NO:45.

198) The polypeptide of claim 197, wherein the polypeptide does not include a transmembrane domain.

199) The polypeptide of claim 194, wherein the polypeptide includes SEQ ID NO:88.

200) The polypeptide of claim 199, wherein the polypeptide does not include a transmembrane domain.

201) A purified SPEX expression vector, comprising: a nucleic acid sequence encoding a SPEX polypeptide operably linked to a vector sequence having one or more control elements for expressing the SPEX polypeptide.

202) The SPEX expression vector of claim 201, wherein the SPEX polypeptide comprises a fusion of a first SPEX amino acid sequence operably linked with a second amino acid sequence.

203) The SPEX expression vector of claim 202, wherein the second amino acid sequence comprises a solubility enhancing polypeptide.

204) The SPEX expression vector of claim 202, wherein the solubility enhancing polypeptide comprises an Ig domain from IgG.

205) The SPEX expression vector of claim 202, wherein the solubility enhancing polypeptide comprises SEQ ID NO:97.

206) The SPEX expression vector of claim 205, wherein the SPEX polypeptide comprises SEQ ID NO:3.

207) of claim 202, wherein the second amino acid sequence comprises a tag.

208) The polynucleotide of claim 187, comprising pEYFP-N1.

209) The polynucleotide of claim 187, wherein the polynucleotide comprises

210) A purified host cell, comprising a SPEX expression vector including a nucleic acid sequence encoding a SPEX expression product operably linked with a vector sequence having one or more control elements for expressing the SPEX expression product.

211) The host cell of claim 210, wherein the SPEX expression product comprises a polynucleotide that is complementary to a nucleic acid polymer encoding a SPEX polypeptide.

212) The host cell of claim 210, wherein the SPEX expression product comprises a SPEX polypeptide.

213) The host cell of claim 210, comprising a bacterial cell.

214) The host cell of claim 210, comprising a yeast cell.

215) The host cell of claim 210, comprising an insect cell.

216) The host cell of claim 210, comprising a mammalian cell.

217) The host cell of claim 210, comprising a human cell.

218) An isolated antibody that immunoreacts with a SPEX polypeptide.

219) The antibody of claim 218, wherein the antibody comprises a monoclonal antibody, or immunoreactive fragment thereof.

220) The antibody of claim 218, wherein the antibody comprises a human monoclonal antibody, a humanized monoclonal antibody, or immunoreactive fragment thereof.

221) The antibody of claim 218, wherein the SPEX polypeptide comprises a human SPEX polypeptide.

222) The antibody of claim 218, wherein the antibody immunoreacts with an extracellular domain of a SPEX polypeptide.

223) The antibody of claim 222, wherein the antibody comprises a monoclonal antibody, or immunoreactive fragment thereof.

224) The antibody of claim 223, wherein the antibody modulates a metabolism of a cell expressing the SPEX polypeptide.

225) The antibody of claim 224, wherein the cell comprises a lymphocyte.

226) The antibody of claim 225, wherein the metabolism comprises a cellular proliferation.

227) The antibody of claim 226, wherein the cellular proliferation is stimulated.

228) The antibody of claim 226, wherein the cellular proliferation is inhibited.

229) The antibody of claim 218, wherein the antibody immunoreacts with an extracellular domain of a SPEX polypeptide, but does not immunoreact with an intracellular domain of a SPEX polypeptide.

230) The antibody of claim 218, wherein the antibody immunoreacts with an intracellular domain of a SPEX polypeptide.

231) The antibody of claim 218, wherein the antibody immunoreacts with an intracellular domain of a SPEX polypeptide, but does not immunoreact with an extracellular domain of a SPEX polypeptide.

232) The antibody of claim 218, wherein the antibody immunoreacts with an amino acid sequence set forth in SEQ ID NO:21, SEQ ID NO:63, or SEQ ID NO:85.

233) The antibody of claim 218, wherein the antibody immunoreacts with an amino acid sequence set forth in SEQ ID NO:7, SEQ ID NO:9, or SEQ ID NO: 11.

234) A method of identifying a SPEX binding partner, comprising:
a) contacting a candidate binding partner with a SPEX polypeptide;
b) detecting whether or not a binding complex is formed that includes the SPEX polypeptide;
c) selecting the candidate binding partner as a SPEX binding partner when the binding complex is formed.

235) The method of claim 234, wherein the SPEX polypeptide comprises SEQ ID NO:3, SEQ ID NO:45, or SEQ ID NO:88.

236) The method of claim 234, wherein the SPEX polypeptide comprises an extracellular domain of the SPEX polypeptide.

237) The method of claim 234, wherein the SPEX polypeptide comprises a fusion polypeptide having a first amino acid including a SPEX Ig like domain operably linked to a second amino acid including a constant region of an IgG polypeptide.

238) The method of claim 234, wherein the SPEX polypeptide includes a detectable label.

239) The method of claim 234, wherein the SPEX polypeptide comprises an intracellular domain of the SPEX polypeptide.

240) The method of claim 239, wherein the SPEX polypeptide includes one or more of SEQ ID NO:7, SEQ ID NO:9, or SEQ ID NO:11.

241) The method of claim 234, wherein the candidate binding partner comprises a polypeptide.

242) The method of claim 234, wherein the candidate binding partner comprises an organic chemical compound.

243) A method of isolating a SPEX polypeptide from a biological sample containing the SPEX polypeptide, comprising:
a) contacting the biological sample with an affinity matrix having an anti-SPEX antibody bound to the matrix, to produce a complex including the SPEX polypeptide and the anti-SPEX antibody bound to the matrix;
b) separating the matrix and the remainder of the biological sample;
c) separating the SPEX polypeptide and the anti-SPEX antibody; and
d) collecting the isolated SPEX polypeptide.

244) A method of modulating a metabolism of a lymphocyte that expresses a SPEX polypeptide, comprising contacting the lymphocyte with an anti-SPEX antibody that immunoreacts with an extracellular domain of the SPEX polypeptide.

245) The method of claim 244, wherein the metabolism comprises a proliferation.

246) The method of claim 245, wherein the proliferation is stimulated.

247) The method of claim 244, wherein the anti-SPEX antibody immunoreacts with an immunoglobulin like domain of the SPEX polypeptide.

248) The method of claim 244, wherein the anti-SPEX antibody immunoreacts with SEQ ID NO:13, SEQ ID NO:55, or SEQ ID NO:85.

249) The method of claim 244, wherein the antibody is a monoclonal antibody or a fragment thereof capable of immunoreacting with an extracellular domain of a SPEX polypeptide.

250) The method of claim 244, wherein the lymphocytes are in an in vitro culture.


SEQUENCE LISTING (113 sequences)

SEQ ID NO: 1 - protein, 30 aa, Homo sapiens
MKTLPAMLGTGKLFWVFFLIPYLDIWNIHG

SEQ ID NO: 2 - protein, 20 aa, Homo sapiens
KESCDVQLYIKRQSEHSILA

SEQ ID NO: 3 - protein, 65 aa, Homo sapiens
GDPFELECPVKYCANRPHVTWCKLNGTTCVKLEDRQTSWKEEKNISFFILHFEPVLPNDNGSYRC

SEQ ID NO: 4 - protein, 42 aa, Homo sapiens
SANFQSNLIESHSTTLYVTDVKSASERPSKDEMASRPWLLYS

SEQ ID NO: 5 - protein, 21 aa, Homo sapiens
LLPLGGLPLLITTCFCLFCCL

SEQ ID NO: 6 - protein, 44 aa, Homo sapiens
RRHQGKQNELSDTAGREINLVDAHLKSEQTEASTRQNSQVLLSE

SEQ ID NO: 7 - protein, 8 aa, Homo sapiens
TGIYDNDP

SEQ ID NO: 8 - protein, 23 aa, Homo sapiens
DLCFRMQEGSEVYSNPCLEENKP

SEQ ID NO: 9 - protein, 9 aa, Homo sapiens
GIVYASLNH

SEQ ID NO: 10 - protein, 14 aa, Homo sapiens
SVIGLNSRLARNVK

SEQ ID NO: 11 - protein, 13 aa, Homo sapiens
EAPTEYASICVRS

SEQ ID NO: 12 - protein, 127 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYS

SEQ ID NO: 13 - protein, 157 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYS

SEQ ID NO: 14 - protein, 148 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCL

SEQ ID NO: 15 - protein, 178 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCL

SEQ ID NO: 16 - protein, 67 aa, Homo sapiens
TGIYDNDPDLCFRMQEGSEVYSNPCLEENKPGIVYASLNHSVIGLNSRLARNVKEAPTEYASICVRS

SEQ ID NO: 17 - protein, 111 aa, Homo sapiens
RRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 18 - protein, 132 aa, Homo sapiens
LLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 19 - protein, 239 aa, Homo sapiens
GDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 20 - protein, 259 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 21 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 22 - DNA, 90 bp, Homo sapiens
atgaagacattgcctgccatgcttggaactgggaaattattttgggtcttcttcttaatcccatatctggacatctggaacatccatggg

SEQ ID NO: 23 - DNA, 60 bp, Homo sapiens
aaagaatcatgtgatgtacagctttatataaagagacaatctgaacactccatcttagca

SEQ ID NO: 24 - DNA, 201 bp, Homo sapiens
ggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgca

SEQ ID NO: 25 - DNA, 120 bp, Homo sapiens
aattttcagtctaatctcattgaaagccactcaacaactctttatgtgacagatgtaaaaagtgcctcagaacgaccctccaaggacgaaatggcaagcagaccctggctcctgtatagt

SEQ ID NO: 26 - DNA, 63 bp, Homo sapiens
ttacttcctttggggggattgcctctactcatcactacctgtttctgcctgttctgctgcctg

SEQ ID NO: 27 - DNA, 132 bp, Homo sapiens
agaaggcaccaaggaaagcaaaatgaactctctgacacagcaggaagggaaattaacctggttgatgctcaccttaagagtgagcaaacagaagcaagcaccaggcaaaattcccaagtactgctatcagaa

SEQ ID NO: 28 - DNA, 24 bp, Homo sapiens
actggaatttatgataatgaccct

SEQ ID NO: 29 - DNA, 69 bp, Homo sapiens
gacctttgtttcaggatgcaggaagggtctgaagtttattctaatccatgcctggaagaaaacaaacca

SEQ ID NO: 30 - DNA, 27 bp, Homo sapiens
ggcattgtttatgcttccctgaaccat

SEQ ID NO: 31 - DNA, 42 bp, Homo sapiens
tctgtcattggactgaactcaagactggcaagaaatgtaaaa

SEQ ID NO: 32 - DNA, 39 bp, Homo sapiens
gaagcaccaacagaatatgcatccatatgtgtgaggagt

SEQ ID NO: 33 - DNA, 381 bp, Homo sapiens
aaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagt

SEQ ID NO: 34 - DNA, 471 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagt

SEQ ID NO: 35 - DNA, 444 bp, Homo sapiens
aaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctg

SEQ ID NO: 36 - DNA, 534 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctg

SEQ ID NO: 37 - DNA, 201 bp, Homo sapiens
actggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 38 - DNA, 333 bp, Homo sapiens
agaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 39 - DNA, 396 bp, Homo sapiens
ttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 40 - DNA, 717 bp, Homo sapiens
ggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 41 - DNA, 777 bp, Homo sapiens
aaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 42 - DNA, 870 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagttaa

SEQ ID NO: 43 - protein, 29 aa, Mus musculus
MKTVPAMLGTPRLFREFFILHLGLWSILC

SEQ ID NO: 44 - protein, 27 aa, Mus musculus
EKATKRNDEECPVQLTITRNSKQSART

SEQ ID NO: 45 - protein, 68 aa, Mus musculus
GELFKIQCPVKYCVHRPNVTWCKHNGTICVPLEVSPQLYTSWEENQSVPVFVLHFKPIHLSDNGSYSC

SEQ ID NO: 46 - protein, 56 aa, Mus musculus
STNFNSQVINSHSVTIHVTERTQNSSEHPLITVSDIPDATNASGPSTMEERPGRTW

SEQ ID NO: 47 - protein, 24 aa, Mus musculus
LLYTLLPLGALLLLLACVCLLCFL

SEQ ID NO: 48 - protein, 37 aa, Mus musculus
KRIQGKEKKPSDLAGRDTNLVDIPASSRTNHQALPSG

SEQ ID NO: 49 - protein, 8 aa, Mus musculus
TGIYDNDP

SEQ ID NO: 50 - protein, 21 aa, Mus musculus
WSSMQDESELTISLQSERNNQ

SEQ ID NO: 51 - protein, 9 aa, Mus musculus
GIVYASLNH

SEQ ID NO: 52 - protein, 14 aa, Mus musculus
CVIGRNPRQENNMQ

SEQ ID NO: 53 - protein, 13 aa, Mus musculus
EAPTEYASICVRS

SEQ ID NO: 54 - protein, 151 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTW

SEQ ID NO: 55 - protein, 180 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTW

SEQ ID NO: 56 - protein, 175 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFL

SEQ ID NO: 57 - protein, 204 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFL

SEQ ID NO: 58 - protein, 65 aa, Mus musculus
TGIYDNDPWSSMQDESELTISLQSERNNQGIVYASLNHCVIGRNPRQENNMQEAPTEYASICVRS

SEQ ID NO: 59 - protein, 102 aa, Mus musculus
KRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 60 - protein, 126 aa, Mus musculus
LLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 61 - protein, 250 aa, Mus musculus
GELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 62 - protein, 277 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 63 - protein, 306 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 64 - DNA, 87 bp, Mus musculus
atgaagacagtgcctgccatgcttgggactcctcggttatttagggaattcttcatcctccatctgggcctctggagcatcctttgt

SEQ ID NO: 65 - DNA, 81 bp, Mus musculus
gagaaagctactaagaggaatgatgaagagtgtccagtgcaacttactattacgaggaattccaaacagtctgccaggaca

SEQ ID NO: 66 - DNA, 210 bp, Mus musculus
ggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctaca

SEQ ID NO: 67 - DNA, 162 bp, Mus musculus
aacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttgg

SEQ ID NO: 68 - DNA, 72 bp, Mus musculus
ctgctttacaccttgcttcctttgggggcattgcttctgctccttgcctgtgtctgcctgctctgctttctg

SEQ ID NO: 69 - DNA, 111 bp, Mus musculus
aaaaggatccaagggaaagaaaagaagccttctgacttggcaggaagggacactaacctggttgatattccagccagttccaggacaaatcaccaagcactgccatcagga

SEQ ID NO: 70 - DNA, 24 bp, Mus musculus
actggaatttatgataatgatccc

SEQ ID NO: 71 - DNA, 63 bp, Mus musculus
tggtctagcatgcaggatgaatctgaattgacaattagcttgcaatcagagagaaacaaccag

SEQ ID NO: 72 - DNA, 27 bp, Mus musculus
ggcattgtttatgcttctttgaaccat

SEQ ID NO: 73 - DNA, 42 bp, Mus musculus
tgtgttattggaaggaatccaagacaggaaaacaacatgcag

SEQ ID NO: 74 - DNA, 39 bp, Mus musculus
gaggcacccacagaatatgcatccatttgtgtgagaagt

SEQ ID NO: 75 - DNA, 453 bp, Mus musculus
gagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttgg

SEQ ID NO: 76 - DNA, 540 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttgg

SEQ ID NO: 77 - DNA, 525 bp, Mus musculus
gagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctg

SEQ ID NO: 78 - DNA, 612 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctg

SEQ ID NO: 79 - DNA, 195 bp, Mus musculus
actggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 80 - DNA, 306 bp, Mus musculus
aaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 81 - DNA, 378 bp, Mus musculus
ctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 82 - DNA, 750 bp, Mus musculus
ggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 83 - DNA, 831 bp, Mus musculus
gagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 84 - DNA, 921 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaa

SEQ ID NO: 85 - protein, 179 aa, Mus musculus
MKTVPAMLGTPRLFREFFILHLGLWSILCEKATKRNDEECEVQLNIKRNSKHSAWTGELFKIECPVKYCVHRPHVTWCKHNGTIWVPLEVGPQLYTSWEENRSVPVFVLHFKPIHLSDNGSYSCSTNFNSQVINSHSVTIHVRERTQNSSEHPLITVSDIPDATNASGPSTMEERPGRT

SEQ ID NO: 86 - protein, 150 aa, Mus musculus
EKATKRNDEECEVQLNIKRNSKHSAWTGELFKIECPVKYCVHRPHVTWCKHNGTIWVPLEVGPQLYTSWEENRSVPVFVLHFKPIHLSDNGSYSCSTNFNSQVINSHSVTIHVRERTQNSSEHPLITVSDIPDATNASGPSTMEERPGRT

SEQ ID NO: 87 - protein, 29 aa, Mus musculus
MKTVPAMLGTPRLFREFFILHLGLWSILC

SEQ ID NO: 88 - protein, 68 aa, Mus musculus
GELFKIECPVKYCVHRPHVTWCKHNGTIWVPLEVGPQLYTSWEENRSVPVFVLHFKPIHLSDNGSYSC

SEQ ID NO: 89 - DNA, 537 bp, Mus musculus
atgaagacagtgcctgccatgcttgggactcctcggttatttagggaattcttcatcctccatctgggcctctggagcatcctttgtgagaaagctactaagaggaatgatgaagagtgtgaagtgcaacttaatattaagaggaattccaaacactctgcctggacaggagagttatttaaaattgaatgtcctgtgaaatactgtgttcatagacctcatgtgacttggtgtaagcacaatggaacaatctgggtaccccttgaagttggtcctcagctatacactagttgggaagaaaatcgatcagttccggtttttgttctccattttaaaccaatacatctcagtgataacgggtcgtatagctgttctacaaacttcaattctcaagttattaatagccattcagtaaccatccatgtgagagaaaggactcaaaactcttcagaacacccactaataacagtatctgacatcccagatgccaccaatgcctcaggaccatccaccatggaagagaggccaggcaggact

SEQ ID NO: 90 - DNA, 450 bp, Mus musculus
gagaaagctactaagaggaatgatgaagagtgtgaagtgcaacttaatattaagaggaattccaaacactctgcctggacaggagagttatttaaaattgaatgtcctgtgaaatactgtgttcatagacctcatgtgacttggtgtaagcacaatggaacaatctgggtaccccttgaagttggtcctcagctatacactagttgggaagaaaatcgatcagttccggtttttgttctccattttaaaccaatacatctcagtgataacgggtcgtatagctgttctacaaacttcaattctcaagttattaatagccattcagtaaccatccatgtgagagaaaggactcaaaactcttcagaacacccactaataacagtatctgacatcccagatgccaccaatgcctcaggaccatccaccatggaagagaggccaggcaggact

SEQ ID NO: 91 - DNA, 87 bp, Mus musculus; identical to SEQ ID NO: 64

SEQ ID NO: 92 - DNA, 204 bp, Mus musculus
ggagagttatttaaaattgaatgtcctgtgaaatactgtgttcatagacctcatgtgacttggtgtaagcacaatggaacaatctgggtaccccttgaagttggtcctcagctatacactagttgggaagaaaatcgatcagttccggtttttgttctccattttaaaccaatacatctcagtgataacgggtcgtatagctgt

SEQ ID NO: 93 - protein, 6 aa, Artificial Sequence
  Xaa = any amino acid
IXYXXL

SEQ ID NO: 94 - protein, 6 aa, Artificial Sequence
  Synthesized
IVYASL

SEQ ID NO: 95 - protein, 6 aa, Artificial Sequence
  Xaa = any amino acid
TXYXXI

SEQ ID NO: 96 - protein, 6 aa, Artificial Sequence
  Synthesized
TEYASI

SEQ ID NO: 97 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 521 aa, Artificial Sequence
  Fusion polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 99 - protein, 538 aa, Artificial Sequence
  Fusion polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 100 - protein, 411 aa, Artificial Sequence
  Fusion polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMKTVPAMLGTPRLFREFFILHLGLWSILCEKATKRNDEECEVQLNIKRNSKHSAWTGELFKIECPVKYCVHRPHVTWCKHNGTIWVPLEVGPQLYTSWEENRSVPVFVLHFKPIHLSDNGSYSCSTNFNSQVINSHSVTIHVRERTQNSSEHPLITVSDIPDATNASGPSTMEERPGRT

SEQ ID NO: 101 - protein, 8 aa, Artificial Sequence
  Synthesized
DYKDDDDK

SEQ ID NO: 102 - protein, 10 aa, Artificial Sequence
  Synthesized
EQKLISEEDL

SEQ ID NO: 103 - protein, 7 aa, Artificial Sequence
  Synthesized
TGIYDND

SEQ ID NO: 104 - DNA, 990 bp, Homo sapiens
actggggtaggtaaactgacccaactctgcagcactcagaagacgaagcaaagccttctacttgagcagtttttccatcactgatatgtgcaggaaatgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagttaagtctgttttctgacctcccaacag

SEQ ID NO: 105 - DNA, 1250 bp, Mus musculus
agatctctagggaggaagaggaagtttgcccttacctgacacgtgctgggaatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtaccca

SEQ ID NO: 106 - DNA, 957 bp, Mus musculus
ggcccgggatctatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagtgcagatccaccggtcgccaccatggtg

SEQ ID NO: 107 - DNA, 1722 bp, Mus musculus
ggatctcgagctcaagcttcgaattctgcagtcgacggtaccgcgggcccgggatctatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagtgcagatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggc

SEQ ID NO: 108 - DNA, 609 bp, Mus musculus
ggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtacccaaagggcgaattctgcagatcagatct

SEQ ID NO: 109 - DNA, 1276 bp, Mus musculus
agatctctagggaggaagaggaagtttgcccttacctgacacgtgctgggaatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtacccaaagggcgaattctgcagatcagatct

SEQ ID NO: 110 - DNA, 486 bp, Mus musculus
ggatcttggagaaagctactaagaggaatggagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggactagatcc

SEQ ID NO: 111 - DNA, 464 bp, Mus musculus
agatcttggagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggactagatct

SEQ ID NO: 112 - DNA, 538 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggactgc

SEQ ID NO: 113 - DNA, 537 bp, Mus musculus; identical to SEQ ID NO: 89